(19) United States
(12) Patent Application Publication
(10) Pub. No.: US 2024/0050908 A1
(43) Pub. Date: Feb. 15, 2024

(54) MICROFLUIDIC PLATFORMS FOR LARGE SCALE NANOPARTICLE FORMULATIONS

(71) Applicant: THE TRUSTEES OF THE UNIVERSITY OF PENNSYLVANIA, Philadelphia, PA (US)

(72) Inventors: Michael J. MITCHELL, Philadelphia, PA (US); David Aaron ISSADORE, Philadelphia, PA (US); Sagar Prasad YADAVALI, Dowingtown, PA (US); Sarah J. SHEPHERD, Philadelphia, PA (US)

(21) Appl. No.: 18/259,439

(22) PCT Filed: Dec. 27, 2021
(86) PCT No.: PCT/US2021/073114

§ 371 Date: Jun. 27, 2023

Related U.S. Application Data
(60) Provisional application No. 63/131,008, filed on Dec. 28, 2020.

(51) Int. Cl. B01F 33/3012; B01F 25/431; B01F 23/41; B01F 23/45; A61K 9/50; A61K 48/00; A61K 39/215

(52) U.S. Cl. CPC B01F 33/3012; B01F 25/43172; B01F 25/431971; B01F 23/4143; B01F 23/4105; B01F 23/45; A61K 9/5089; A61K 48/0041; A61K 39/215; B01F 2101/22

(57) ABSTRACT

Provided are scalable, parallelized microfluidic chips that include arrays of microfluidic mixing channels for large-scale production of lipid nanoparticles, among other products. The disclosed chips can operate with a single set of inlets and outlet, and achieve production rates in excess of those achieved by existing methods. The disclosed devices provide large-scale production of formulations while still maintaining the physical properties and potency typical of existing methods of producing such formulations. Also provided are related methods of using the disclosed devices.

### RELATED APPLICATIONS

The present application claims priority to and the benefit of U.S. patent application No. 63/131,008, “Microfluidic Platform For Large Scale RNA Lipid Nanoparticle Formulations” (filed Dec. 28, 2020), the entirety of which application is incorporated herein by reference for any and all purposes.

### GOVERNMENT RIGHTS

This invention was made with government support under TR002776 awarded by the National Institutes of Health. The government has certain rights in the invention.

### TECHNICAL FIELD

The present disclosure relates to the field of microfluidics and to the field of lipid nanoparticle formation.

### BACKGROUND

Lipid nanoparticles (LNPs) are a promising delivery technology that have enabled clinical translation of RNA therapeutics. However, a key challenge toward the broad clinical translation of LNP-based RNA therapeutics and vaccines is the development of formulation strategies that can robustly produce precisely defined formulations while accommodating scalable throughputs ranging from early development to clinical translation. Microfluidic approaches have demonstrated control over physical properties not possible with conventional bulk methods, but such approaches are limited by scalability challenges and their inherently low production rates (<100 mL/hr). Accordingly, there is a long-felt need in the field for scalable systems and methods for production of LNPs and other nanoparticles, which systems and methods can be used at comparatively higher production rates.

### SUMMARY

To address this challenge, we present a scalable, parallelized microfluidic device (PMD) that incorporates an array of 128 microfluidic mixing channels (4×32) for large-scale production of LNPs with a single set of inlets and outlet that achieves >100× production rates compared to single microfluidic channels. Compared to small scale (single microfluidic channels) and large scale (bulk mixing) LNP formulation strategies, we show that our PMD produces large-scale LNP formulations while still maintaining the physical properties and potency typical of microfluidic-generated LNPs. In mice, we show that LNPs encapsulating either siRNA targeting Factor VII or luciferase-encoding mRNA generated using our scalable PMD are superior to LNPs generated using bulk mixing methods, with a four-fold increase in hepatic gene silencing and five-fold increase in luciferase expression, respectively. Collectively, these results suggest that this PMD has the potential to generate scalable and reproducible LNP formulations that are needed for emerging clinical applications, including RNA therapeutics and vaccines such as those in development for COVID-19.

In meeting the described long-felt needs, the present disclosure provides a microfluidic chip, comprising: a first supply channel configured to communicate a first fluid therein in a direction from upstream to downstream; a plurality of first delivery channels in fluid communication with the first supply channel; a second supply channel configured to communicate a second fluid therein in a direction from upstream to downstream; a plurality of second delivery channels in fluid communication with the first supply channel, a number of rows comprising a number of mixing device units, a mixing device unit comprising (i) a micromixer channel, (ii) a first flow resistor placing the micromixer channel of that mixing device unit into fluid communication with a first delivery channel associated with that mixing device unit, and (iii) a second flow resistor placing the micromixer channel of that mixing device unit into fluid communication with a second delivery channel associated with that mixing device unit; and an output channel, the output channel being in fluid communication with at least one of the mixing device units, and the output channel being configured to collect an output of at least one of the mixing device units.

Also provided are methods, comprising operating a microfluidic chip according to the present disclosure so as to give rise to an encapsulating product.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1A-1C provide example fabrication of a scalable PDMS-based microfluidic platform for precise and large-scale RNA lipid nanoparticle formulations. (FIG. 1A) Microfluidic formulation produces smaller and more homogeneous LNPs for potent RNA delivery, while larger and more heterogeneous LNPs produced by bulk methods are more variable in terms of RNA delivery. Microfluidic formulation can be scaled up using the same design (staggered herringbone micromixers, or SHM)) for rapid mixing to produce comparable LNPs for RNA therapy. (FIG. 1B) A photograph of the PDMS device, which incorporates 128 (4×32) mixing channels with only two inlets and one outlet. Scale bar: 5 mm. (FIG. 1C) Production rate comparison of different continuous LNP formulation methods (T-junction¹⁻⁵; hydrodynamic focusing^{6,7}; SHM^{8,9}; NanoAssemblr Ignite, NxGen Blaze, GMP system¹⁰), highlighting the total volumetric production rate and respective LNP size.

FIGS. 2A-2F illustrate incorporation of ladder design architecture, flow resistors and herringbone micromixers enable large-scale LNP production. (FIG. 2A) Schematic diagram for the design of this device containing 4 rows of 32 mixing channels, highlighting the individual unit design with a top view and a side view. Schematic is not to scale. (FIG. 2B) A circuit model of the delivery channels (R_{delivery}), resistors (Rᵣₑₛᵢₛₜₒᵣ), mixing channels (R_{mixing}), and an individual mixing channel unit (R_{device}). (FIG. 2C) The resistance of an individual mixing channel unit versus length of the mixing channel, where the R_{device} is dominated by the resistance of the fluidic resistors, not the mixing channels. R_{device} is normalized to the resistance of an individual mixing channel unit with resistors and a mixing channel length of 20 cycles. (FIG. 2D) A cross section of the PMD. Scale bar: 200 μm. (FIGS. 2E-2F) Device features. Scale bars: 200 μm.

FIGS. 3A-3J provide validation of uniform mixing across all scales of microfluidic devices. Mixing characterization for microfluidic devices: single channel (A-B), single row PMD (C-F), PMD (G-J). (FIG. 3A) Schematic for the mixing experiment, where FITC dextran and rhodamine B dextran were flowed through a single channel device to quantify mixing at various channel lengths, showing the red green plot profile versus channel distance at the outlet (inset). The architecture of a single cycle of staggered herringbone micromixers is shown. Scale bar: 200 μm. (FIG. 3B) Quantification of mixing versus channel length for five different flow rates, where the channel length for 90% mixing (±standard error mean) is directly correlated with the natural log of the Peclet number (inset). (FIG. 3C) Schematic for the mixing experiment with a single row PMD of 10 parallel channels. (FIG. 3D-3E) Fluorescent images of mixing for a channel, showing the red-green plot profile versus channel distance at the outlet (inset). Scale bars: 200 μm. (FIG. 3F) Comparison of the channel length required for 90% mixing (±standard error mean) for all 10 channels and the single channel device. Samples were compared by one-way ANOVA. ns: p>0.05. (FIG. 3G) Schematic for the mixing experiment with the PMD. (FIGS. 3H-3I) Fluorescent images of mixing for a channel, showing the red green plot profile versus channel distance at the outlet (inset). (FIG. 3J) Comparison of the channel length required for 90% mixing (±standard error mean) for 3 channels across the device and the single channel device. Samples were compared by one-way ANOVA. ns: p>0.05.

FIGS. 4A-4F provide validation of mixing, uniform LNP physical properties, and potent RNA delivery in vitro across all scales of microfluidic devices. (FIG. 4A) Ten mixing channels are organized in a single row, with the two inlets connected in a ladder geometry to each channel, showing the beginning (left), center (middle), and end (right) of each channel. Scale bars: 200 μm (FIG. 4B) Quantification of the ratio of red to green inlets (±standard deviation) for each channel. (FIG. 4C) DLS curves for firefly luciferase siRNA LNPs produced from each channel. (FIG. 4D) Luciferase expression (±standard deviation) in HeLa cells after treatment with 5 nM of luciferase siRNA LNPs produced from each channel. Data is normalized to cells without treatment with background subtracted. N=10. (FIG. 4E) A photograph of the single row PMD with two inputs (blue, orange dye) and 10 outputs, one for each mixing channel, used to collect LNPs from each channel. Scale bar: 5 mm. (FIG. 4F) The calculated IC50 (±standard deviation) indicates the luciferase siRNA dose needed to silence 50% of the firefly luciferase gene for each formulation method. Samples were compared by one-way ANOVA. ns: p>0.05.

FIGS. 5A-5D illustrate that the disclosed scalable PMD formulates LNPs with uniform physical properties for potent in vivo therapeutic RNA delivery compared to bulk mixing methods. (FIG. 5A) Schematic for comparison between microfluidic single channel device, single row PMD, and bulk mixing. (FIG. 5B) Microfluidic-formulated LNPs encapsulating siRNA to knockdown Factor VII were delivered to C57BL/6 mice to show the sustained knockdown over time and to determine the optimal dose and collection timepoint. LNPs were formulated with Factor VII siRNA by a microfluidic single channel device, and Factor VII activity was quantified as a percentage of activity 1 day prior to LNP administration at days 2, 7, 15, and 21 post injection. n=3. (FIG. 5C) Mice were dosed with 0.2 mg/kg of Factor VII siRNA LNPs, and Factor VII activity was quantified 48 hours later. Factor VII activity is reported as a percentage of activity 1 day prior to LNP administration. **: p<0.01 (p=0.0038); ****: p<0.0001 in Dunnett's multiple comparisons test to siControl LNP. n=4. (FIG. 5D) LNPs were formulated with mRNA encoding firefly luciferase and administered to mice via tail vein injection at a dose of 0.2 mg/kg. Luminescent flux was quantified 4 hours after LNP administration for n=3-4 replicates with two representative animals shown below. ***: p<0.001 in Dunnett's multiple comparisons test to bulk mixed LNPs. n=3-4.

FIGS. 6A-6C provide an illustrative design of PMD and validation of uniform fabrication. (FIG. 6A) Schematic for side profile of each unit in the PMD, highlighting layer heights for each feature. Schematic is not to scale. (FIGS. 6B-6C) Validation of uniform channel heights across the single row PMD (FIG. 6B) and PMD (FIG. 6C). Device was cut vertically with a razor blade and channel height was quantified by microscopy. Desired height was 70 μm. n=3. Samples were compared by one-way ANOVA. ns: p>0.05.

FIGS. 7A-7B provides a characterization of single stranded DNA (ssDNA) LNPs formulated by the single row PMD indicates uniform performance for each mixing channel. (FIG. 7A) ssDNA LNPs were formulated at a variety of flow rates per channel (0.1-1.26 mL/min), where LNP size (±standard deviation) was dependent on flow rate until the smallest size (50 nm) LNPs were formed. This experiment was repeated with a microfluidic single channel device for comparison. n=3. (FIG. 7B) ssDNA LNPs were formulated with the single row PMD with individual outputs, and hydrodynamic size (±standard deviation) was quantified by DLS. n=3. Samples were compared by one-way ANOVA. ns: p>0.05.

FIGS. 8A-8E illustrate that siRNA LNPs targeting firefly luciferase were formulated in the single row PMD with individual outputs. LNP size, knockdown efficiency in vitro, and cell viability were quantified for 1-10 nM siRNA doses. (FIG. 8A) Particle size (±standard deviation) was quantified for LNPs formulated by each channel of the PMD, microfluidic single channel device, or bulk mixing by DLS. (FIG. 8B) Cell viability (±standard deviation) was quantified by a CellTiter-Glo kit for cells treated with 10 nM (max dose) firefly luciferase siRNA LNPs. (FIG. 8C) Dose response for HeLa cells treated with 1-10 nM firefly luciferase siRNA LNPs, showing luciferase expression (±standard deviation) normalized to untreated cells with background subtracted. N=10. (FIG. 8D) Example IC50 calculation for microfluidic single channel LNPs, where luciferase expression is plotted versus siRNA concentration, and an exponential decay curve determines the siRNA concentration for 50% expression. (FIG. 8E) Calculated IC50 (±standard deviation) from the data in (C) for each channel and alternative formulation methods. Samples were compared by one-way ANOVA. ns: p>0.05.

FIGS. 9A-9E illustrate that microfluidic-formulated LNPs encapsulating Factor VII siRNA or luciferase mRNA had a small size and high encapsulation efficiency compared to bulk formulation. (FIG. 9A) A photograph of the PMD with different colored dyes (blue, orange) for each inlet. Scale bar: 5 mm. (FIGS. 9B, 9D) Tables reporting the diameter (DLS z-average), polydispersity index (PDI), RNA concentration, and encapsulation efficiency (±standard deviation) for each formulation of either Factor VII siRNA LNPs (FIG. 9B) or firefly luciferase mRNA LNPs (FIG. 9D). (FIGS. 9C, 9E) Dynamic light scattering (DLS) curves showing the intensity distribution (±standard deviation) of either Factor VII siRNA LNPs (FIG. 9C) or firefly luciferase mRNA LNPs (FIG. 9E) for the different formulation methods.

FIG. 10 illustrates that mouse liver histopathology indicates no significant differences in toxicity between LNP formulations. C57BL/6 mice were dosed with 0.2 mg/kg Factor VII siRNA LNPs, and liver samples were examined 48 hours after LNP administration. Representative images of hematoxylin and eosin staining are shown for each experimental group at 20× (left) and 40× (right) magnification. Minor mixed lymphocytic infiltrates (circles) and individual necrotic hepatocytes (arrows) were observed in all groups and determined to be incidental and not secondary to test article. Scale bars: 100 μm (left panel), 50 μm (right panel).

FIGS. 11A-11C illustrate that liver toxicity assays indicate no significant differences in toxicity between LNP formulations. C57BL/6 mice were dosed with 0.2 mg/kg luciferase mRNA LNPs, and liver enzymes were quantified 4 hours after injection. (FIG. 11A) Aspartate transaminase (AST) quantification (±standard deviation) for the formulation groups and controls. n=3-4. (FIG. 11B) Alanine transaminase (ALT) quantification (±standard deviation) for the formulation groups and controls. n=3-4. (FIG. 11C) Alkaline phosphatase (ALP) quantification (±standard deviation) for the formulation groups and controls. n=3-4. Reference ranges are listed for C57BL/6 mice for each enzyme (FIGS. 11A-11C).

FIG. 12 provides an annotated version of FIG. 2 to highlight the various flow resistances in an exemplary device.

FIG. 13 provides a schematic for each mixing unit in an example PMD, highlighting example layer heights for each layer (bottom side view). Direction of flow is indicated by white arrows. Schematic is not to scale.

FIGS. 14A-14G illustrate the incorporation of ladder design architecture, flow resistors and herringbone micromixers to enable large-scale LNP production. (FIGS. 14A-14C) Schematic diagram for the design of this device containing 4 rows of 32 mixing channels. (FIG. 14A), highlighting the individual mixing unit design with a top view and a side view (FIG. 14B) and the individual mixing cycle design with a top, angled, and side view (FIG. 14C). Direction of flow is indicated by white arrows. Schematics are not to scale. (FIG. 14D) A circuit model of the delivery channels (R_{delivery}), resistors (Rᵣₑₛᵢₛₜₒᵣ) mixing channels (R_{mixing}), and an individual mixing channel unit (R_{device}). (FIG. 14E) The resistance of an individual mixing channel unit versus length of the mixing channel, where the R_{device} is dominated by the resistance of the fluidic resistors, not the mixing channels. R_{device} is normalized to the resistance of an individual mixing channel unit with resistors and a mixing channel length of 20 cycles. (FIG. 14F) Device features. Scale bars: 200 μm. (FIG. 14G) A cross section of the PMD. Scale bar: 200 μm.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints, 2 grams and 10 grams, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

A major unmet need in the advancement of lipid nanoparticles (LNPs) for RNA therapeutics is the development of formulations that can be produced reliably across the various scales of drug development, ranging from small-scale discovery experiments to large-scale clinical trials. Microfluidic technologies, which generate LNPs with precisely defined properties, have been limited by their scalability challenges. To overcome this, we developed a new microfluidic architecture that has a production rate that can be scaled across phases of drug development without sacrificing the desirable LNP physical properties and potency of microfluidic generated LNPs. This technology can be widely applied to accelerate LNP-based RNA therapeutics or vaccines to address emerging pathogens and sudden outbreaks, such as the ongoing COVID-19 pandemic.

RNA therapeutics—a potent and versatile alternative to pharmacological drugs such as small molecules and proteins—treat illnesses by modifying the expression of disease-related genes that are considered ‘undruggable’ with current medicines.^{11,12} These disease-related genes can be controlled depending on the type of RNA: small interfering RNA (siRNA) or micro RNA (miRNA) for gene knockdown, messenger RNA (mRNA) for transient gene expression, or a combination of guide RNA (gRNA) and mRNA for CRISPR/Cas9-related gene editing.^{5,13-16} Recent advances in RNA therapeutics led to the U.S. Food and Drug Administration approval of the lipid nanoparticle (LNP)-based siRNA therapeutic Onpattro (Alnylam Pharmaceuticals, Inc.) and the approval of the N-acetylgalactosamine (GalNAc) siRNA conjugate Givlaari (Alnylam Pharmaceuticals, Inc.), in addition to several ongoing phase 3 clinical trials for COVID-19 mRNA vaccines.¹⁷⁻²⁴ Since naked RNAs are subject to rapid degradation by serum endonucleases and cannot readily cross cell membranes due to charge repulsions, RNA therapeutics require a delivery platform for intracellular delivery.^{41,25,26} Thus, synthetic delivery vehicles—such as LNPs—have been developed for RNA delivery due to their biocompatibility, potent intracellular delivery, as well as protection from innate immune recognition and endonuclease degradation.^{14,27-29} Additionally, LNPs have the versatility to encapsulate a single type or multiple types of RNA, and formulations are easily tunable to optimize the lipid and nucleic acid components for the desired application.³⁰⁻³³ While LNPs are well-suited for RNA therapeutics and vaccines, a key challenge toward their broad clinical translation is the development of formulation techniques that can robustly and consistently produce formulations on production scales that range from early phases of development to clinical applications.

LNPs can be formulated by several methods, which can be grouped into macroscopic and microscale processes. Generally, all of these methods mix a lipid solution dissolved in ethanol and a nucleic acid solution to produce LNPs via self-assembly. For each of these methods, LNP size is an important parameter to control since it greatly influences particle fate in vivo.³⁴ Two main examples of macroscopic processes are the preformed vesicle method and pipette mixing. Although these macroscopic processes require less equipment for LNP formulations, both processes lack precise control over mixing time and thus create large (>100 nm), polydisperse particles that vary batch-to-batch and can require more downstream processing in large-scale manufacturing.^{9,15,35} As an alternative to these processes, rapid microfluidic mixing processes are designed for complex folding of fluids in microseconds to milliseconds, which allows more precise size control, more homogenous solutions, higher encapsulation efficiencies and greater reproducibility.^{8,36} An example of this is hydrodynamic flow focusing, a microfluidic laminar flow method where particles are formed at the interface of the input streams—however, this process is highly limited in throughput and mainly used for preparing liposomes, which have less structural complexity than LNPs.^{6,7,37,38} Another type of rapid mixing process is T-junction mixing, a method of LNP production that uses turbulent rapid mixing in a macroscopic channel (>1 mm) requiring very high flow rates for production (>40 mL/min).^{2,8,15,32} LNPs produced from T-junction mixing are typically larger (>130 nm) than those produced by microfluidic methods, and this method is not scalable down to the small volumes (μL) desired for high-throughput library screening of nanomaterials.^{8,33,34}

One strategy for the production of these smaller volumes is staggered herringbone micromixer designs that induce chaotic mixing in microchannels, which have been widely applied to LNP production.^{8,9,39} This design enables laminar flows of reagents to be rapidly and controllably mixed (<10 ms) at low Reynolds numbers, with high reproducibility, LNP size homogeneity, and with lowest reported LNP sizes (<30 nm).^{8,9,36} However, while the staggered herringbone micromixer design achieves favorable LNP physical properties, this technology is still limited by low flow rates (<100 mL/hr) that are not suitable for clinical scales (L/hr). Notably, the success of this microfluidic architecture has led to commercialization by Precision Nanosystems, Inc., entitled the NanoAssemblr™ platform, which has commercialized devices with the staggered herringbone micromixer design and a toroidal mixer design—where series of bifurcating mixers induce LNP self-assembly.^{10,33} While these instruments have achieved clinically relevant production rates (>20 L/hr), it requires multiple devices operating independently.

Despite these efforts, there remains an unmet need for a single integrated design that can formulate nanoparticles (e.g., nucleic acid therapeutics) in a controlled manner on production scales ranging from benchtop to human applications for LNPs and other nanoparticles to reach their full clinical potential. Adding additional relevance to this challenge, for LNP-based COVID-19 mRNA vaccines, the formulation of LNPs has been identified as the bottleneck for the production process.⁴⁰

We address this challenge by developing an exemplary parallelized microfluidic device (PMD) that can incorporate an array of 1×-128×SHM mixing channels that operate simultaneously, allowing throughput to be scaled from mL/hr to L/hr without sacrificing the desirable LNP physical properties and potency typical of microfluidic-generated LNPs. To this end, we present a polydimethylsiloxane (PDMS) PMD with an array of SHMs (1×, 10× and 128×) and only a single set of inlets and outlet for throughput-invariant formulation of LNPs encapsulating RNA therapeutics. This technology is directly scalable from small-scale discovery experiments (μL) to clinically-relevant production rates (L) by simply integrating more mixing units into a single microfluidic chip, eliminating the need for additional optimizations or further scale-up challenges. To address this challenge and to ensure that each device in the array produces LNPs with identical physical parameters, we incorporated a ladder geometry that incorporates flow resistors upstream of each SHM to distribute fluid evenly to each SHM in the array. Moreover, the flow resistors decoupled the design of the mixing channels and the design requirements for parallelization, allowing the modular incorporating of mixing channel geometries into our architecture without redesign. While parallelized microfluidic architectures have been implemented for NP production, these have been limited in throughput and cannot be scaled to the thousands of devices necessary for operation at the commercial scale. Thus, there is need for a single integrated device that can formulate nucleic acid therapeutics in a controlled manner on production scales ranging from benchtop to human applications in order for LNPs to reach their full clinical potential.

Here, we present, inter alia, a monolithic polydimethylsiloxane (PDMS) parallelized microfluidic device (PMD) with an array (4×32) of 128 mixing channels with only a single set of inlets and outlet for large-scale formulation (L/hr) of LNPs encapsulating RNA therapeutics. Although the PDMS device is used to illustrate the disclosed technology, it should be understood that this example design is illustrative only and does not limit the scope of the present disclosure or the appended claims. The disclosed design has some relation to the field of droplet microfluidics, where architectures and fabrication processes have been developed to operate tens to thousands of identical channels at once on the same device in both PDMS elastomer/glass and silicon/glass substrates.^{41,42}

The disclosed technology, however, differs from existing efforts in a number of nonobvious ways. As a first example, the disclosed technology effects chaotic mixing, and chaotic mixing is not necessarily suitable for production of emulsions. As a second example, the presently disclosed technology can comprise features (e.g., channels, mixers, and other features) that have a cross-sectional dimension that is on the order of tens to hundreds of micrometers. Such a scale, however, differs from the comparatively small scale (e.g., 10-20 micrometers) seen in existing microfluidic efforts. Due to the increased feature size, chaotic mixers have relatively low fluidic resistance compared to emulsion architectures; thus, alternative microfluidic strategies (e.g., flow resistors) must be employed to abide by fluidic requirements necessary for parallelization.

To ensure that each device in the exemplary array produces LNPs with identical physical parameters, we incorporated a ladder geometry with small channels that increase fluidic resistance—flow resistors—to effect certain design rules.^{43,44} For our PMD with 128 mixing channels operating simultaneously, we quantified mixing using fluorescent dyes at a total flow rate of 3.2 L/hr, demonstrating equal mixing efficiency to the current small-scale single channel microfluidic device. We compared three formulation methods—microfluidic single channel, PMD, and bulk mixing—to produce LNPs that encapsulate either siRNA for gene knockdown in vitro and in vivo or mRNA for gene expression in vivo. PMD formulation resulted in LNPs with comparable physical properties to those generated using single channel microfluidics, both of which produced smaller LNPs than those produced by bulk mixing. We demonstrated superior performance of LNPs formulated by both microfluidic methods in vivo for the reduction of Factor VII activity in C57BL/6 mice, where we achieved 90% reduction in activity at 0.2 mg/kg while bulk mixed LNPs only reduced activity by 20%. Similarly, we demonstrated superior performance of LNPs formulated by both microfluidic methods in vivo for luciferase mRNA delivery, where we achieved a five-fold increase in luciferase expression compared to bulk mixed LNPs at a dose of 0.2 mg/kg. The fabrication of this PMD addresses the clinical need of scalable LNP production, with the potential to enable rapid formulation of reproducible and homogenous LNPs for a broad range of RNA therapeutics and vaccines.

Results

Fabrication of Scalable, Parallelized Microfluidic Device

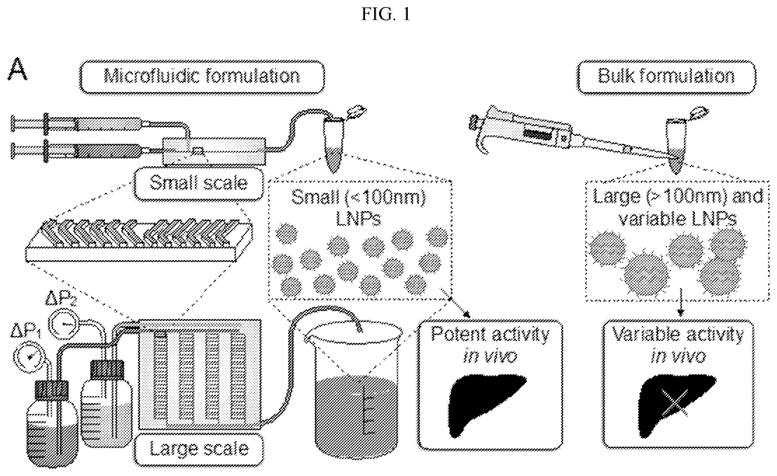

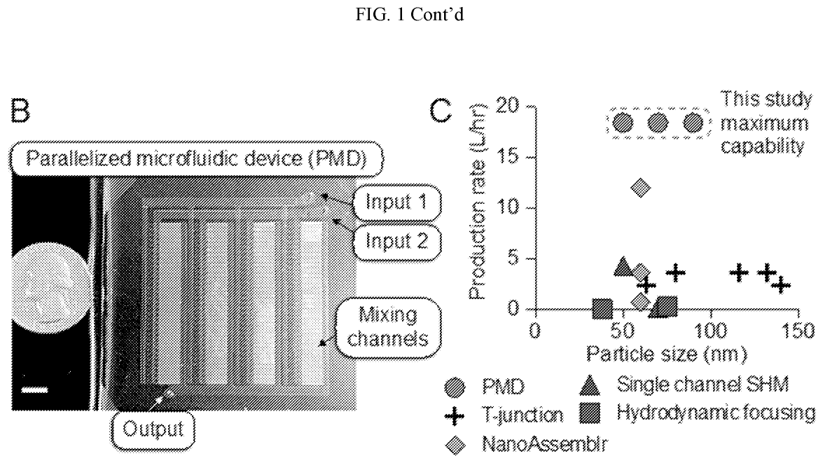

We have fabricated a PMD for large-scale manufacturing of LNPs that increases production rates by over 100-fold (18.4 L/hr) compared to the current single channel microfluidic device.⁹ By incorporating a ladder design architecture, flow resistors, and soft lithography techniques, we have patterned our designs into a single piece of PDMS that can be operated at moderate pressures (50 psi) without delamination.⁴⁵ PDMS was selected for the device material due to its low cost, widespread use in the field, and compatibility with the intended solvents.^{46,47} Our design and fabrication enable production of monodisperse LNPs—with a polydispersity index (PDI) less than 0.2—encapsulating siRNA or mRNA for in vitro and in vivo delivery studies. By incorporating the staggered herringbone micromixer geometry shown by others,^{9,48} we take advantage of the already demonstrated benefits of microfluidic LNP production, such as reproducible production of small size LNPs (<100 nm) and low polydispersity (FIG. 1A). Compared to a method of bulk LNP production, such as repeated pipetting to mix solutions, microfluidic devices produce LNPs with higher potency for gene silencing or gene expression in vivo (FIG. 1A).

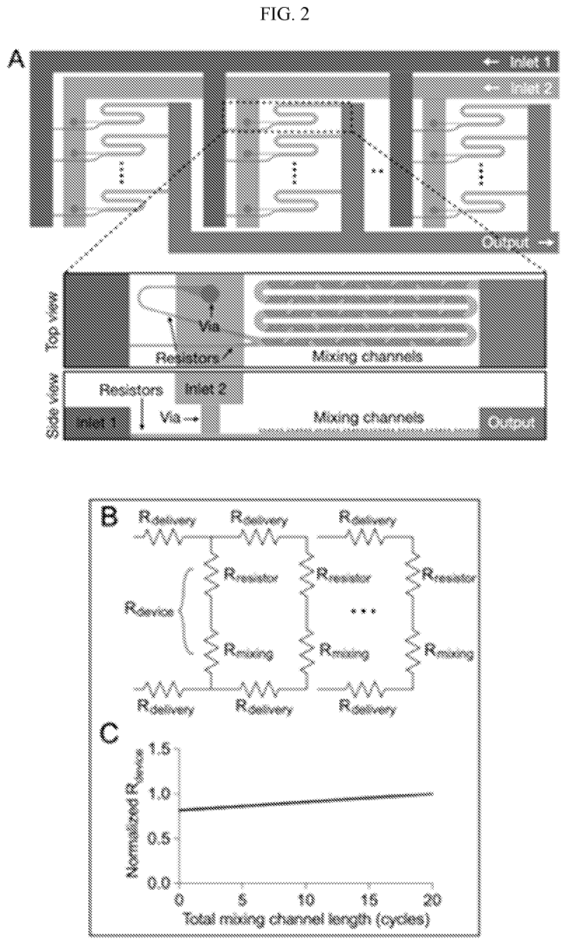

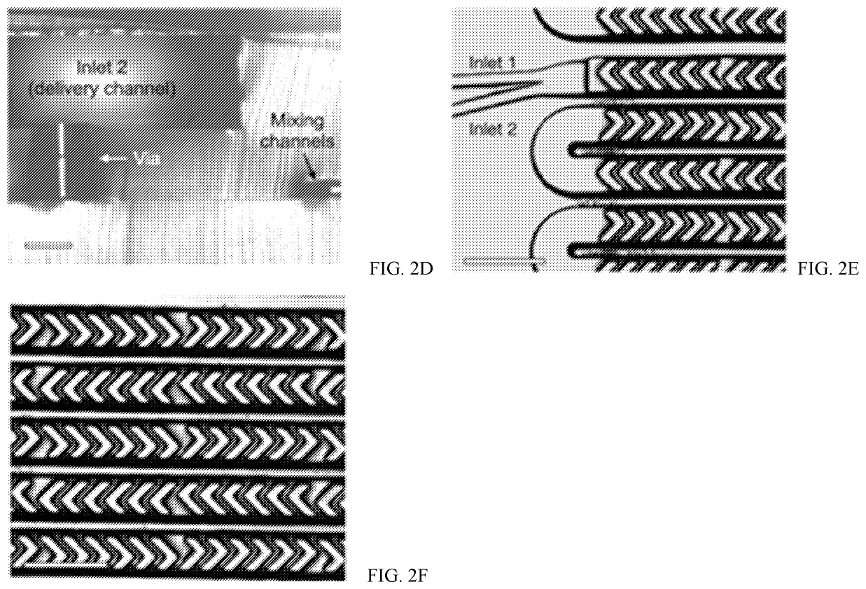

Our PMD (FIG. 1B) consists of an array of chaotic micromixers, each connected to layers of channels that deliver and collect fluid from each device in the array. The PMD is designed to incorporate 128 mixing channels in a ladder architecture to ensure that each device in the array operates with the same flow conditions (FIG. 2A). The micromixer devices are arranged in an array of 4 rows of 32, covering an area of 4.5 cm by 5 cm. In our ladder design, supply channels connect to the array using 4 rows of delivery channels, each connected by a single arterial line that connects to the external inlets and outlets. Upstream of each chaotic micromixers are flow resistors (FIG. 2E), designed with sufficient fluidic resistance such that the flow is distributed uniformly across all of the micromixers using certain design rules.^{41,43} Each micromixer device is connected to the second layer of channels (in a plane above the mixing channels) by a vertical via (FIG. 2A, 2D). The device also contains microfabricated in-line filters (20 μm and 50 μm) to remove any debris that could clog the microscale flow channels.

Microfluidic Design Principles

For uniform operation of each of the chaotic micromixer in the array, the fluidics are designed such that each micromixer in the array receives its inputs with the same flow rates. To achieve this goal, one can design a device wherein each chaotic mixer is designed such that the fluidic resistance of each micromixer device is much greater than that of the delivery channels, allowing the micromixers to operate as if they are connected in parallel.^{43,44} However, because we developed this technique to be modular such that it can be applied to any particle-generating device at any level of parallelization, we did not want to have the parallelization considerations place any restrictions on the design of the individual generators. To this end, we incorporated flow resistors upstream of each inlet to each micromixer (FIG. 2E-F), which dominate the overall resistance of the device, thus decoupling the design of the mixing channels and its requirements for parallelization. The resistance of a single mixing device is the sum of the resistance of flow resistors (Rᵣₑₛᵢₛₜₒᵣ) and the resistance of micromixer channels (R_{mixing}) to make a total device resistance (R_{device}) (FIG. 2B), which is dominated by the resistance of the fluidic resistors (FIG. 2C). Using these flow resistors to set the device flow resistance, we were able to incorporate a specific number of devices per row N_{dev} using a design rule that relates the number of devices allowed per row (N_{dev}) to the resistance of delivery channels and resistance of devices:⁴³

2N_{device}(R_{delivery}/R_{device})<0.01.

The fluidic resistance of the rectangular channels in our device were estimated as Rαμl/wh³, in the condition where h<w, and where μ is the fluidic viscosity, l is the length, w is the width, and h is the height of the channel. In our design, the maximum number of allowable channels per row was N_{device}=112, and we conservatively included 32 devices per row. Additionally, our PMD had to satisfy the design constraint to ensure that each row received the same flow rate from the arterial delivery channel—a constraint that is related to the resistance of the supply channel between delivery channel rows (R_{supply}) and the resistance of each row (R_{row}). We approximated the resistance of each row by dividing the resistance of each device by the number of devices per row (R_{row}=R_{device}/N_{device}) such that each row would be considered N_{device}=32 mixing channels in parallel. Thus, the design rule for choosing the number of rows was:

2N_{row}(R_{supply}/R_{row})<0.01.

By this constraint, in our design we could have a maximum number of rows N_{row}=6, and we conservatively designed our PMD with 4 delivery channel rows.

Validation of Parallelized Microfluidic Design

To validate our parallelized microfluidic design, we performed mixing analysis by flowing two different fluorescent dyes through the device and quantifying a mixing value for various flow rates and channel positions. The two fluorescent dyes, fluorescein isothiocyanate (FITC) dextran and rhodamine B dextran, were selected since they could be detected by different filters via fluorescence microscopy, and the intensity of each fluorescent stream could be quantified independently.

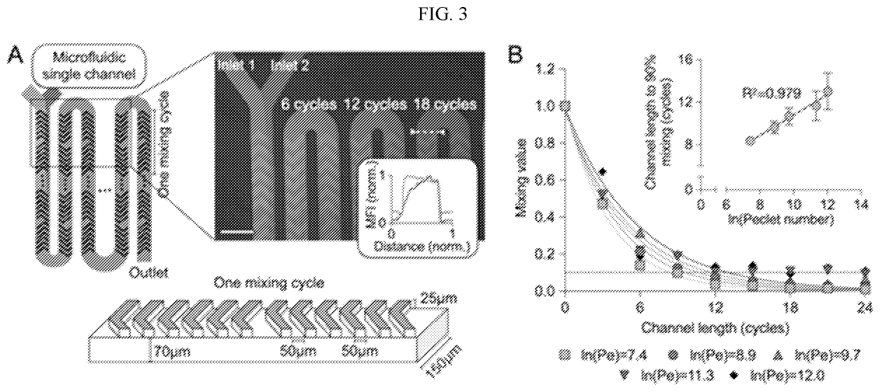

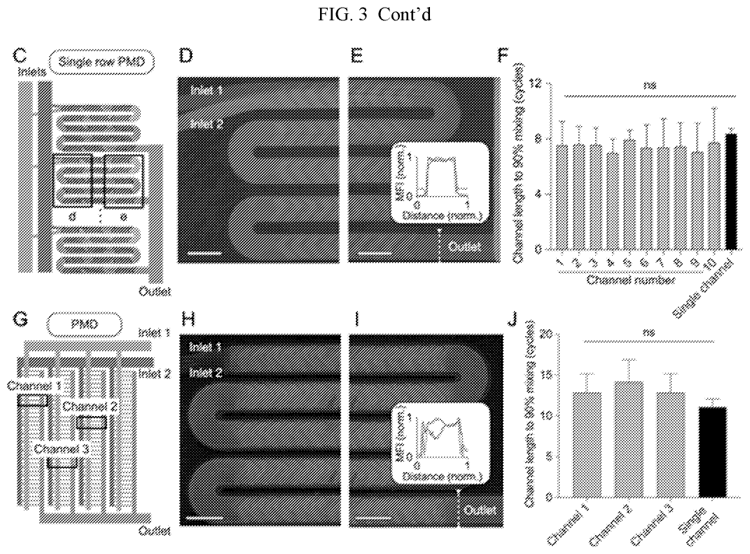

We fabricated a single channel device with cycles of staggered herringbone micromixers to evaluate mixing efficiency at different flow rates (FIG. 3A). We flowed the two fluorescent dyes at a 3:1 flow rate ratio through the device and quantified a mixing value ranging from 1 (not mixed) to 0 (completely mixed) (detailed in Materials and Methods). We calculated this mixing value for five different flow rates—which are indicated by their respective Peclet number, a dimensionless quantity that is directly proportional to total volumetric flow rate (FIG. 3B). For each flow rate, we determined the channel length at which the dyes are 90% mixed (mixing value=0.1)—a value that could be compared between devices to evaluate mixing performance. As validated in other studies, we obtained a linear relationship between the natural log of the Peclet number and channel length needed for 90% mixing (FIG. 3B inset), which is a main advantage of rapid mixing using staggered herringbone micromixers.³⁹ Additionally, this property is maintained over two orders of magnitude of flow rates (24 μL/min to 2400 μL/min).

In addition to the PMD with 128 channels, we fabricated a single row PMD with only 10 mixing channels to validate our design principles. Using the single row PMD, we quantified the channel length needed for 90% mixing using FITC dextran and rhodamine B dextran and compared it to the single channel device at the same flow rate per channel (FIG. 3C-F). Similarly, we used the PMD with 128 channels to quantify the channel length needed for 90% mixing and compared it to the single channel device at the same flow rate per channel (FIG. 3G-J). Our mixing quantification for the single row PMD and PMD demonstrates that mixing efficiency is unaffected by the incorporation of 10 or 128 mixing channels, validating that our device was properly designed.

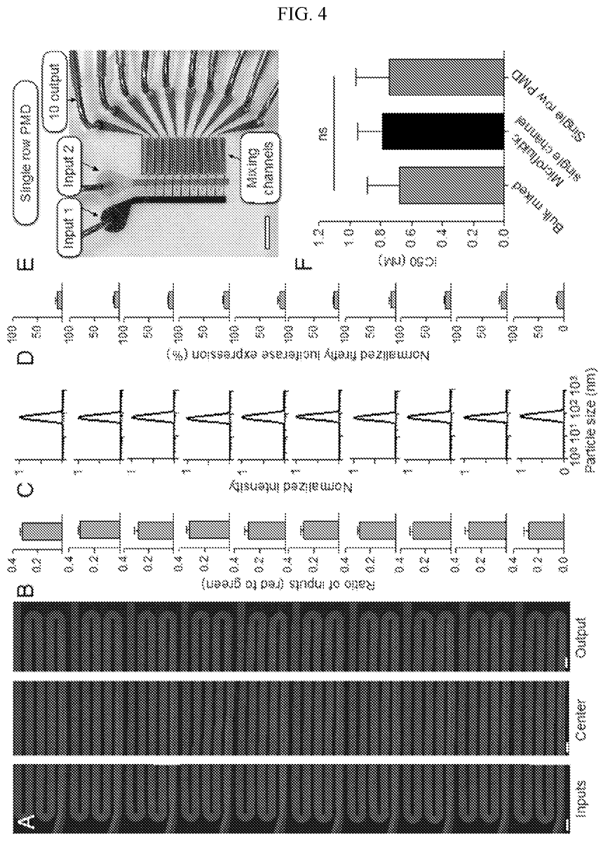

To ensure our device was fabricated uniformly with no variation between mixing channels, thus producing comparable LNPs in each channel, we quantified channel heights and the flow rate ratio for each channel. By evaluating the cross sections of each channel, we determined there were no differences in channel heights across the device (FIG. 6). To quantify the flow rate ratios for each of the channels of the single row PMD, we flowed two fluorescent dyes through the device and imaged each of the 10 channels at three different positions (FIG. 4A). For LNP formulations, it is critical to control the flow rate ratio (lipids to nucleic acids), thus we quantified the ratio of rhodamine B to FITC (red to green dye) for each channel (FIG. 4B) and found no significant differences between channels. Here, we show that each channel has a flow rate ratio of ˜0.3 of red to green dye, which is the ratio of the lipids to nucleic acids needed for LNP formulations.²⁴ Collectively, we validated the uniform fabrication and performance of the PMD to ensure it was suitable for formulating LNPs encapsulating RNA for therapeutic applications.

Formulation of LNPs Encapsulating RNAs with Parallelized Device

To validate that our PMD could formulate LNPs at a large scale without compromising desirable physical parameters of LNPs produced by microfluidic single channels, we produced LNPs and evaluated their physical properties—such as size, size distribution, and encapsulation efficiency. Further, to verify that our PMD does not compromise LNP efficacy, we formulated LNPs encapsulating siRNA for in vitro screening in cell lines, as well as LNPs encapsulating either siRNA or mRNA for in vivo gene silencing or gene expression, respectively, in mice. Gene silencing in vitro was evaluated by a robust screening assay^{31,32,49,50} where HeLa cells—modified to stably express firefly (Photinus pyralis) luciferase—were transfected with siRNA LNPs targeting the firefly luciferase gene. To evaluate LNP efficacy in vivo, we formulated LNPs with siRNA to knockdown clotting Factor VII and administered them by intravenous injection to C57BL/6 mice, where the reduction in Factor VII activity could be measured in plasma.^{15,31} Additionally, to evaluate LNP efficacy for mRNA delivery in vivo, we formulated luciferase-encoding mRNA LNPs and administered them by intravenous injection to C57BL/6 mice, where LNP efficacy could be easily determined by luminescent signal.³⁰ Throughout these in vitro and in vivo studies, we demonstrate that potency is comparable between LNPs formulated by a microfluidic single channel device or PMD.

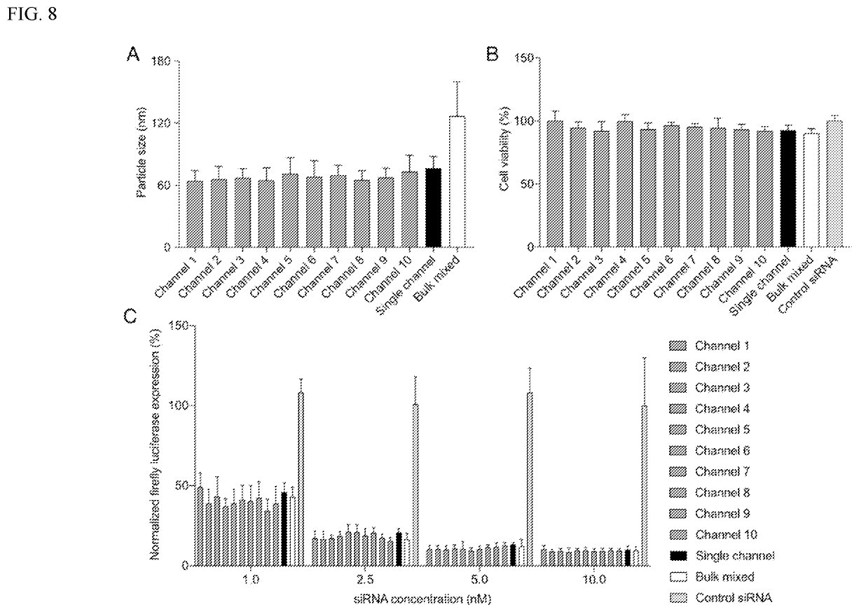

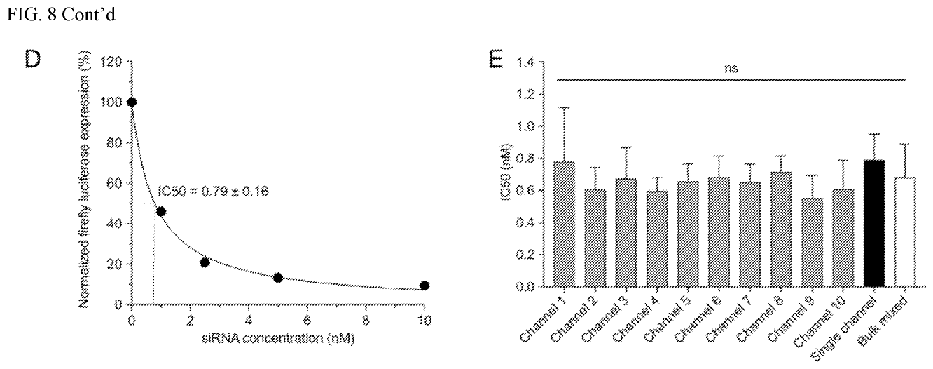

To confirm that LNP formulations were reproducible across all channels of the single row PMD, we fabricated a device with individual outputs for each of the 10 mixing channels for individual channel analysis of LNP formulations (FIG. 4E). We first determined the optimal flow rate conditions by formulating LNPs encapsulating non-coding single stranded DNA (ssDNA) and comparing sizes between the microfluidic single channel device and single row PMD (FIG. 7). To evaluate gene knockdown efficiency in vitro for LNPs formulated by each mixing channel, we formulated siRNA LNPs targeting firefly luciferase to verify that each channel formulated LNPs with similar properties and potency. LNPs were formulated with the ionizable lipid C12-200, a gold standard lipid that is well validated for siRNA and mRNA delivery, to aid in cellular uptake as well endosomal escape to ultimately enable potent intracellular delivery.^{30,51-54} To induce LNP self-assembly in the device, firefly luciferase siRNA was rapidly mixed with a solution of lipids: C12-200, the phospholipid 1,2-dioleoyl-sn-glycero-3-phosphoethanolamine (DOPE), cholesterol, and lipid-PEG. We selected these excipients based on their use in previous RNA delivery studies.^{30,54} Dynamic light scattering (DLS) of the siRNA LNPs from each of the 10 output channels demonstrated no significant differences in size or size distribution (FIG. 4C; FIG. 8). HeLa cells that stably express firefly luciferase were transfected with these siRNA LNPs, and the resultant reduction in firefly luciferase expression (>80%) was comparable between LNPs formulated across the 10 mixing channels (FIG. 4D; FIG. 8). Additionally, firefly luciferase siRNA LNPs were formulated by the single row PMD, a single channel microfluidic device, and bulk mixing, where both microfluidic methods produced small (70 nm) LNPs and bulk mixing produced large (130 nm) LNPs (FIG. 8). HeLa cells were transfected with LNPs generated by each of the formulation methods, and firefly luciferase expression was quantified and compared to untreated cells at four different doses: 1 nM, 2.5 nM, 5 nM, 10 nM (FIG. 8). From this dose response, an IC50 value was calculated for each formulation that indicates the siRNA dose needed for 50% knockdown (FIG. 4F). For these in vitro experiments, knockdown of firefly luciferase was equivalent for all formulation methods tested.

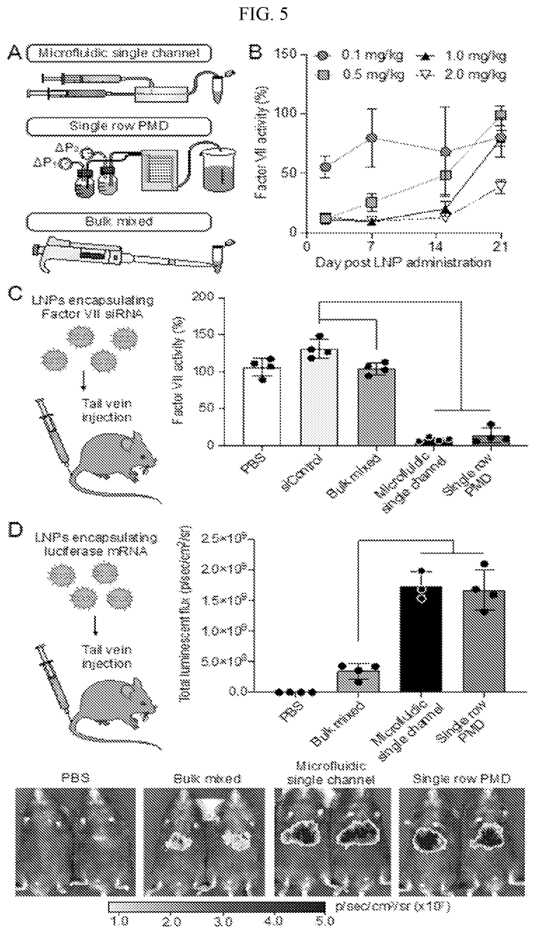

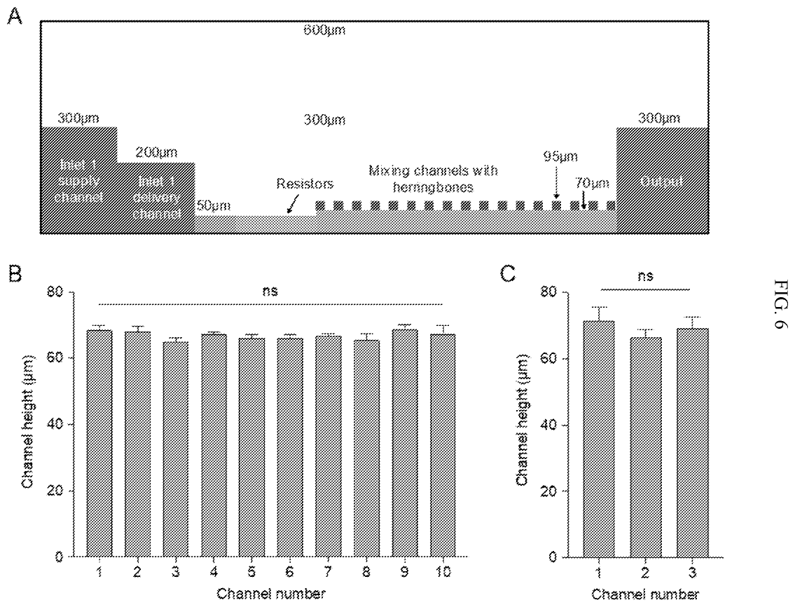

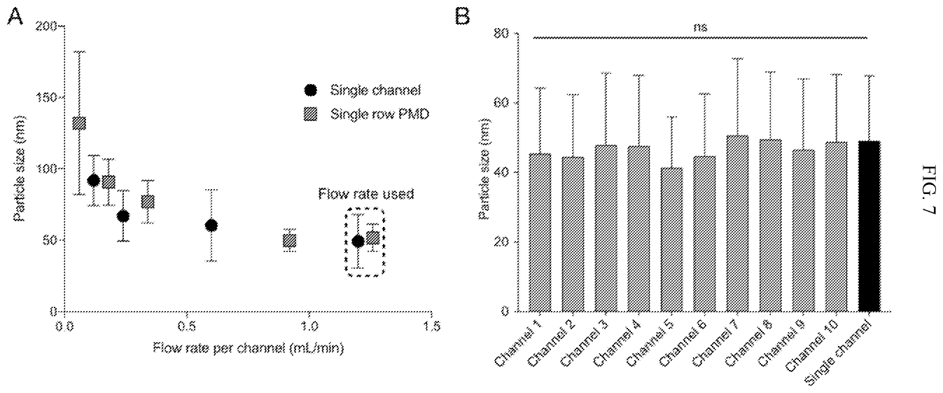

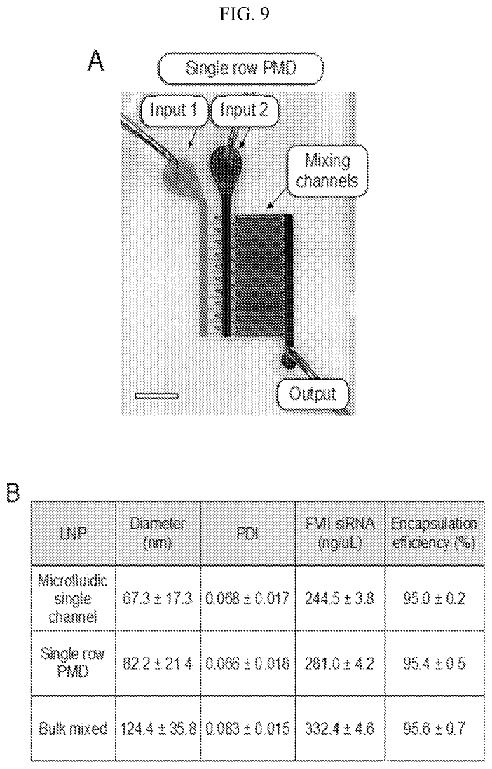

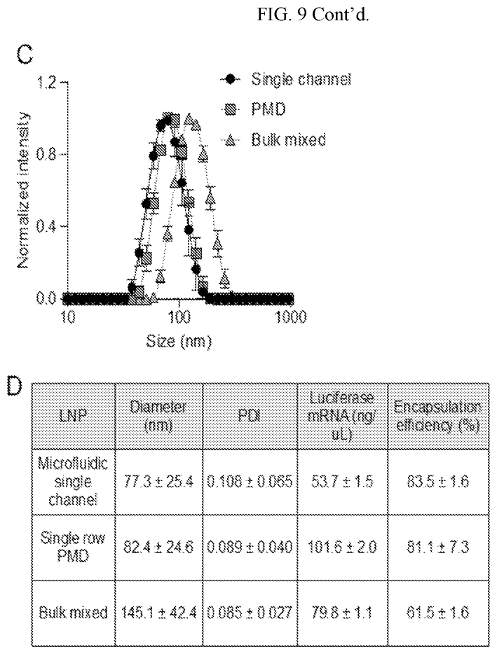

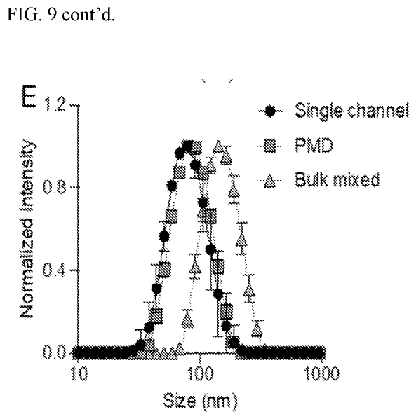

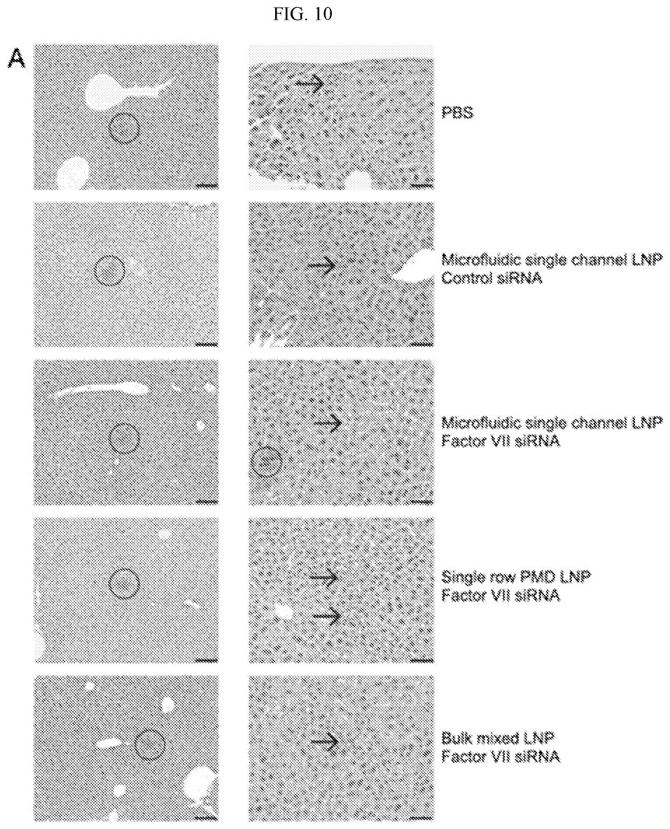

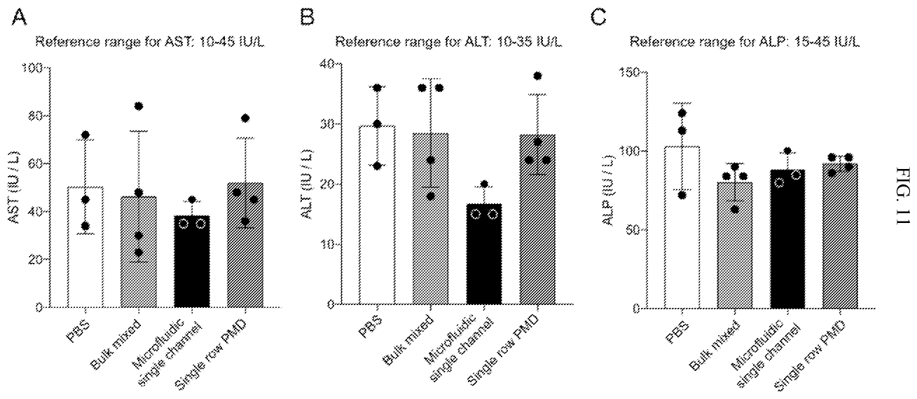

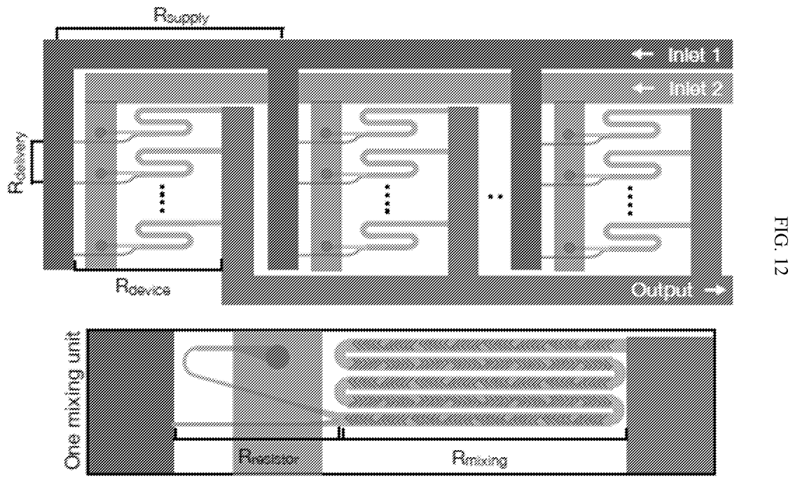

To demonstrate potent in vivo siRNA and mRNA delivery via PMD-formulated LNPs, siRNA LNPs to knockdown Factor VII and luciferase-encoding mRNA LNPs were compared to LNPs produced by the microfluidic single channel device and bulk mixing techniques. siRNA LNPs were formulated by mixing Factor VII siRNA with a solution of lipids: C12-200, 1,2-distearoyl-sn-glycero-3-phosphocholine (DSPC), cholesterol, and lipid-PEG. These excipients were selected based on their use in previous in vivo studies for Factor VII knockdown.^{30,31} Similarly, mRNA LNPs were formulated by mixing luciferase mRNA with a solution of lipids: C12-200, DOPE, cholesterol, and lipid-PEG, where excipients and excipient ratios were selected based on previous studies for mRNA LNP optimization.³⁰ An initial study with siRNA LNPs formulated by the microfluidic single channel device investigated a range of doses (0.1-2.0 mg/kg) for Factor VII knockdown, where a dose of 0.2 mg/kg would achieve >80% knockdown of Factor VII activity in plasma at 2 days post injection (FIG. 5B). Both microfluidic methods produced Factor VII siRNA LNPs with a small size (<85 nm), whereas bulk mixing produced LNPs with a large size (>120 nm) (FIG. 9). Factor VII siRNA LNPs were administered to C57BL/6 mice via tail vein injection, and Factor VII activity was quantified 48 hours after injection (FIG. 5C). PMD and single channel microfluidics produced LNPs that were more potent than bulk mixed LNPs, where microfluidic LNPs reduced Factor VII activity by >90% while bulk mixed LNPs minimally (20%) reduced Factor VII activity. There were no significant differences in toxicity between formulation groups as indicated by histological analysis performed by hematoxylin and eosin (H&E) staining of liver samples, where no histopathological lesions were found in any of the LNP treated animals beyond the incidental finding found in the PBS treatment group (FIG. 10). To show that our technology could also be used for mRNA therapies, we formulated luciferase mRNA LNPs with the three formulation methods, where both microfluidic methods produced LNPs with a small size (<85 nm) and bulk mixing produced LNPs with a large size (>140 nm) (FIG. 9). These mRNA LNPs were administered to C57BL/6 mice via tail vein injection, and luminescence was quantified 4 hours post LNP administration (FIG. 5D-E). LNPs produced by both microfluidic methods were more potent than bulk mixed LNPs, where microfluidic-formulated LNPs demonstrated a five-fold increase of luciferase expression compared to bulk mixed LNPs at a dose of 0.2 mg/kg. Additionally, we observed no clinically significant changes of aspartate transaminase (AST), alanine transaminase (ALT), or alkaline phosphatase (ALP) between formulation groups (FIG. 11). Overall, these results demonstrate that our PMD produces LNPs at a large scale that achieve potent in vivo siRNA and mRNA delivery that are comparable to single channel microfluidics.

Discussion

Here, we have developed a simple and scalable PMD fabricated using PDMS and glass to produce highly monodisperse LNPs on a scale relevant for clinical application, which is >100× the throughput of a single microfluidic device. 9 By using double-sided imprinting to cast multi-layer designs on a monolithic device and incorporating upstream flow resistors to decouple the design of the mixing channels and the overall resistance of the device, we fabricated devices that are inexpensive and can be tailored to the applications of any user. To the authors' knowledge, this is the highest potential flow rate by any published or commercially available single device for LNP production (FIG. 1C). While alternative methods like T-junction mixing can produce LNPs at clinically-relevant rates (2.4-3.6 L/hr), this method generally produces large (>100 nm) LNPs^{1,3,4} that can require higher doses for effective gene therapy. Additionally, the NanoAssemblr™ platform can also achieve clinically relevant rates (>20 L/hr) with desirable LNP properties' but can require multiple devices operating separately. Overall, our scalable PMD addresses the key unmet need of scalable microfluidic processes for LNP formulation.

With emerging opportunities for LNP-based RNA therapeutics and vaccines, scalable processes that can rapidly produce nanomedicines must be implemented. While this study is focused on LNP formulations for gene therapy, other scalable processes have been implemented for NP synthesis. Particle Replication in Nonwetting Templates (PRINT), a layer-by-layer assembly process, can formulate polymeric NPs for a range of shapes and sizes down to 50 nm.^{55,56} Additionally, applications in droplet microfluidics have been used to formulate inorganic NPs for therapeutic and imaging applications, where droplets act as microreactors to facilitate precise, multi-step reactions and could be scaled up by implementing design principles presented in this work.^{57,58} In the context of LNPs, these processes should enable production at small scales (μL) needed for screening to large scales (L) needed for clinical trials without changing LNP physical parameters or activity. Impressively, in response to the ongoing COVID-19 pandemic, Moderna, Inc. manufactured their LNP vaccine mRNA-1273 for phase 1 clinical trials less than 2 months after the SARS-CoV-2 genome was posted in January 2020.²³ To further gene therapy applications, we must continue to develop platforms that can readily scale therapeutics from early development to clinical application. The development of scalable microfluidics, including this study and the NanoAssemblr™ platform, will enable LNP and nanomedicine therapies to become more widely exploited throughout the field as major manufacturing challenges are being addressed.

If alternative solvents or higher pressures are needed for other rapid mixing applications using this architecture, the device can be fabricated out of alternative substrates, such as silicon/glass, that could allow for high temperatures (T>500° C.) and further increase throughput by allowing high pressures (P>1000 psi).³¹ One limitation for the current PMD is the pressure needed to operate the device at its highest flow rate (18 L/hr) which is 286 psi. Our current experimental set up and fabrication technique operated the PMD at a maximum pressure of 50 psi, with a maximum total output of 3.2 L/hr. To produce LNPs at higher pressures (>200 psi), a device can be fabricated out of silicon/glass with established fabrication techniques^{41,42} and similar architecture to achieve these highest flow rates. Our PMD design—one that allows uniform flow distribution without changing the single channel architecture by incorporating flow resistors—can be applied to any microfluidic architecture that needs higher throughput and can be completed with common, inexpensive materials such as PDMS. Total flow rate can also be increased by simply adding more devices to the array, where the maximum number of devices that can be incorporated is determined by flow resistor dimensions, which can be altered arbitrarily.

Here, we have validated our design principles with the PMD and a single row PMD that can rapidly mix two inputs on different scales (128× and 10×, respectively) and produce siRNA LNPs that are potent for gene knockdown in vitro and in vivo, as well as mRNA LNPs that are potent for gene expression in vivo. To validate the device fabrication and operation, we have quantified mixing efficiency, channel dimensions, and the ratio of inputs for each mixing channel across the PMD. We have shown equivalent knockdown of firefly luciferase expression in HeLa cells at low concentrations where LNPs produced by both microfluidic methods and bulk mixing demonstrate equal potency with minimal toxicity. However, when LNPs containing either Factor VII-silencing siRNA or luciferase-encoding mRNA were administered to C57BL/6 mice, both microfluidic methods produced LNPs that were significantly more potent than bulk mixed LNPs. We predict that the poor performance of the bulk mixed LNPs could be attributed to its large size (>120 nm) that led to rapid blood clearance by the reticuloendothelial system and limited passage through liver fenestrations.^{59,60} While microfluidic parameters (i.e. flow rates) can be adjusted to achieve specific LNP sizes⁹ (FIG. 7) depending on the intended application, bulk mixing does not provide consistent means to alter the size of LNPs produced, which is a determining factor in LNP success. Although the microfluidic-formulated LNPs and bulk mixed LNPs differ in terms of their performance in vivo, the three formulation methods all showed minimal toxicity by liver enzymes and histopathological analysis. Overall, the PMD produces potent LNPs that are comparable in physical parameters and in vivo activity to the microfluidic single channel device, while LNPs produced by bulk mixing are larger in size and demonstrate poor in vivo efficacy. These results suggest that this PMD can generate scalable and potent LNP formulations for clinical applications, including RNA therapeutics and vaccines.

Materials and Methods

Device Fabrication

The fabrication strategy for the example PMD used double-sided imprinting,⁴⁵ a technique that enables fabrication of 3D structures by combining a multi-layer negative photoresist (SU-8) hard master with a PDMS-based soft master. Standard photolithography processes were used to fabricate the multi-layer SU-8 masters, where SU-8 was spin-coated to the desired thicknesses on a silicon wafer (University Wafer), followed by UV exposure on a mask aligner (SUSS MicroTec) for each layer using a 365 nm long-pass filter. For the PMD (128 mixing channels), five mask layers were designed for the hard master: filters and resistors (layer 1—height 50 μm), mixing channels (layer 2—height 20 μm), herringbones (layer 3—height 25 μm), delivery channels (layer 4—height 100 μm), supply channels and vias (layer 5—height 200 μm). For the soft master, two mask layers were designed: supply channels (layer 1—height 350 μm) and filters (layer 2—height 50 μm). For the single row PMD, three mask layers were designed for the hard master: filters, resistors and mixing channels (layer 1—height 70 μm), herringbones (layer 2—height 25 μm), vias and delivery channels (layer 3—height 100 μm). For the single row PMD soft master, two mask layers were designed: delivery channels (layer 1—height 130 μm), and filters (layer 2—height 70 μm). The PDMS soft masters were prepared by pouring a 10:1 mixture of PDMS elastomer to curing agent (Thermo Fisher) on the Si masters.

To apply the double-sided imprinting technique to fabricate the PMDs, the soft masters must be silanized with trichloro(1H,1H,2H,2H-perfluorooctyl)silane (MilliporeSigma) following plasma treatment to prevent adhesion of the PDMS soft master to the central PDMS piece. PDMS is poured onto the silanized soft and hard masters, and the designs are aligned using support patterns (2.5 mm×2.5 mm for single row PMD; 3 mm×3 mm for PMD) that are incorporated to the design of soft and hard masters. Once alignment is complete, pressure is applied using five 0.5 kg weights to ensure that the hard master vias maintain contact with the supply channels of the soft master and the PDMS is cured on a 65° C. hotplate for two hours. The 3D designed PDMS layer is bonded to another piece of PDMS using plasma treatment, input/output holes are punched using a 1 mm biopsy punch (Thermo Fisher), and the backside of the hole-punched PDMS double-layer device is bonded to a glass slide using plasma treatment. For the single channel device, two mask layers were designed: mixing channels (layer 1—height 70 μm) and herringbones (layer 2—height 25 μm). Devices were fabricated by curing PDMS on the Si master, hole punching with a 1 mm biopsy punch (Thermo Fisher) and bonding to a glass slide using plasma treatment.

Experimental Setup

A pressure-driven flow system was used to conduct all experiments for the PMDs. Nitrogen pressure tanks were connected to either 100 mL or 1 L Duran pressure plus glass bottles (MilliporeSigma) using PTFE tubing (McMaster-Carr). Pressure regulators (Global Test Supply, LLC) and pressure gauges (Grainger, Inc.) were used to monitor pressures for each inlet, and total volumetric flow rate was measured at the output collection of the device. Microscopy was used to monitor PMD performance and ensure a 3:1 flow rate ratio of nucleic acid input to lipid input. For the single channel device, a Pump 33 DDS (Dual Drive System) (Harvard Apparatus) powered two glass syringes (Hamilton Company) for the nucleic acid and lipid phases at a 3:1 flow rate ratio.

Mixing Characterization

FITC-dextran and rhodamine B isothiocyanate-dextran (MilliporeSigma) were dissolved to 10 μM in water. These solutions were flowed through the single channel device, single row PMD, and PMD at a 3:1 flow rate ratio (FITC to rhodamine B) at various flow rates—where the total volumetric flow rate ranged from 24 μL/min to 2400 μL/min per channel. Fluorescence images of the channel were taken at different channel lengths and the intensity profile of each dye across the channel was quantified using ImageJ (NIH) before being exported to MATLAB (MathWorks) for numerical analysis. Fluorescence intensity of FITC and rhodamine B were processed by subtracting the background, normalizing the intensity, and calculating the area under the curve for the difference of the rhodamine B normalized intensity from the FITC normalized intensity generating a mixing value that ranged from 1 (not mixed) to 0 (completely mixed). Thus, mixing was plotted as a function of channel length, and values were normalized to a mixing value of 1 at the beginning of the channel (0 cycles). The channel length needed for 90% mixing was calculated based on the intersection of an exponential decay model and a mixing value of 0.1. Errors reported are the standard error of the exponential decay model.

Lipid Nanoparticle Formulation and Characterization

LNPs were formulated by rapid mixing of an aqueous phase containing RNA and an ethanol phase containing cholesterol and lipids using a microfluidic device or by pipette mixing. The ionizable lipid C12-200 was synthesized as previously described.^{32,54} For the in vitro experiments, a formulation of C12-200, 1,2-dioleoyl-sn-glycero-3-phosphoethanolamine (DOPE) (Avanti Polar Lipids, Inc.), cholesterol (MilliporeSigma) and 1,2-dimyristoyl-sn-glycero-3-phosphoethanolamine-N-[methoxy(polyethylene glycol)-2000] (PEG2000-PE) (Avanti Polar Lipids, Inc.) were combined at molar ratios of 50%, 10%, 38.5%, and 1.5%, respectively. For the in vivo siRNA experiments, a formulation of C12-200, 1,2-distearoyl-sn-glycero-3-phosphocholine (DSPC), cholesterol, and PEG2000-PE was combined at molar ratios of 50%, 10%, 38.5%, and 1.5%, respectively. For the in vivo mRNA experiments, a formulation of C12-200, DOPE, cholesterol, and PEG2000-PE were combined at molar ratios of 35%, 16%, 46.5%, and 2.5%, respectively. LNPs formulated by the single row PMD had reagents diluted by 2× or 3× to preserve reagents. The C12-200:RNA weight ratio was 10:1 for in vitro siRNA experiments, 5:1 for in vivo siRNA experiments, and 10:1 for in vivo mRNA experiments.

Anti-firefly luciferase siRNA (Dharmacon) was used for in vitro experiments. Anti-Factor VII siRNA (MilliporeSigma) was custom ordered with a sense sequence of 5′-GGAucAucu-cAAGucuuAcT*T-3′ and an anti-sense sequence of 5′-GuAAGAcuuGAGAuGAuccT*T-3′, where asterisks indicate phosphorothioate linkages and lowercase nucleotides are 2′-fluoro-modified. ssDNA LNPs were formulated using C12-200, DOPE, cholesterol, and PEG2000-PE at molar ratios of 35%, 16%, 46.5%, and 2.5%, respectively with a C12-200:DNA weight ratio of 10:1. ssDNA was purchased from IDT with a sequence of A*G*A*CGTGTGCTCTTCCGATCTGAGGGTACTNNNNNNNNNNAGATCGGAA GAGCGTCG*T*G*T, where asterisks indicate phosphorothioate linkages and N indicates randomly generated nucleotides. Firefly luciferase mRNA was synthesized as previously described.⁶¹

Using the syringe pump system for the single channel device or the pressure-driven flow system for PMDs, a 3:1 flow rate ratio of aqueous to ethanol phases was used for nanoprecipitation of LNPs. Flow rate per channel ranged from 0.06 mL/min to 1.26 mL/min total volumetric flow rate. LNPs were then dialyzed against 1×PBS for two hours before filtration by 0.22 μm filters. For in vivo experiments, LNPs were concentrated using 50K MWCO centrifugal filters following dialysis (MilliporeSigma). For the pipette mixed LNPs, an electronic pipette (Eppendorf, Thermo Fisher) mixed the reagents at the maximum flow rate. After dialysis in 1×PBS for two hours, LNPs were filtered by 0.45 μm filters. For characterization, LNPs were diluted 1:100 in 1×PBS and dynamic light scattering (DLS) was performed by a Zetasizer Nano (Malvern Instruments) to measure the hydrodynamic diameter (z-average) and polydispersity index (PDI) in triplicate. Standard deviation of the particle size was calculated by: σ=\sqrt{PDI·diameter²}. RNA concentration was quantified by absorbance at A260 using a Tecan NanoQuant Plate (Thermo Fisher). Encapsulation efficiency was calculated by a Quant-iT RiboGreen (Thermo Fisher) assay. Briefly, LNPs were either diluted in TE buffer as a control, or Triton X-100 (MilliporeSigma) to lyse the LNPs and were plated with RNA standards. The RiboGreen reagent was added, and fluorescence was measured by a plate reader. A standard curve was generated to quantify RNA content, and encapsulation efficiency (%) was calculated as follows: 100*(total RNA−unencapsulated RNA)/(total RNA).

siRNA Delivery to HeLa Cells In Vitro

HeLa cells, stably modified to express firefly luciferase, were cultured in Dulbecco's Modified Eagles Media (DMEM) (Thermo Fisher) supplemented with 10% fetal bovine serum and 1% penicillin/streptomycin. The cells were incubated at 37° C. in 5% CO₂ and plated at a density of 10,000 cells per well (Thermo Fisher). Cells were treated with 1-10 nM siRNA 24 hours after plating, and firefly luciferase expression was measured 24 hours after LNP transfection using a Luciferase Assay System (Promega) according to the manufacturer's protocol. The luminescent signal was normalized to untreated cells after subtracting the background (wells without cells with reagents). To assess cytotoxicity, a CellTiter-Glo Luminescent Cell Viability Assay (Promega) was performed according to manufacturer's instructions, and luminescence was normalized to untreated cells after subtracting the background.

In Vivo LNP Studies

All animal procedures were performed on male C57Bl/6 mice aged 6-8 weeks (The Jackson Laboratory) in accordance with protocols approved by the Institutional Animal Care and Use Committee of the University of Pennsylvania. Mice were administered a single 0.1 mL intravenous dose of formulated LNPs encapsulating siRNA or mRNA in PBS pH 7.4 (Gibco) via tail vein injection. Plasma was obtained by collecting blood in tubes containing a solution of 3.2% sodium citrate at a ratio of 9:1 and spun at 2500×g for 10 minutes. Factor VII protein levels were analyzed by a chromogenic enzyme activity assay (Biophen FVII, Aniara) according to the manufacturer's protocol. A standard curve was constructed using commercially available normal mouse plasma (Sigma) and relative Factor VII expression was determined by comparing treated animals to the corresponding pre-treatment sample. Bioluminescence imaging was performed with an IVIS Spectrum imaging system (Caliper Life Sciences). Mice were administered D-luciferin (PerkinElmer) at a dose of 150 mg/kg by intraperitoneal injection (IP) and anesthetized 5 minutes later by ketamine/xylazine (IP). Image acquisition occurred 20 minutes after D-luciferin administration using an exposure time of 30 seconds with medium binning. Bioluminescence values were quantified by measuring photon flux in the region of interest where bioluminescence signal emanated using the Living IMAGE Software provided by Caliper. At the terminal time point mice were anesthetized by IP injection of ketamine/xylazine and blood was collected via cardiac puncture. Subsequently, mice were euthanized by cervical dislocation and the left lobe of the liver was collected and fixed in 10% neutral buffered formalin (Fisher Healthcare). Blood collection at all other time points was performed by submandibular bleeding. Hematoxylin and eosin staining were performed on sections from paraffin-embedded liver samples according to standard protocols. Liver function tests were performed by Antech and were compared to previously published reference ranges.⁶²

Statistical Analysis

Statistical tests were performed in Graphpad Prism 8. Data were plotted as mean±standard deviation unless otherwise specified. Differences between groups were determined by unpaired t test to an appropriate control or one-way analysis of variance (ANOVA) (specified in figure legends).

The foregoing description is illustrative only and does not limit the scope of the present disclosure or the appended claims. As but one example, although PDMS was used as the material for the exemplary devices described herein, the disclosed technology is not limited to PDMS devices. For example, the disclosed technology can be implemented in, e.g., glass, silicon, and other substrates. Chips according to the present disclosure can be formed using soft lithography, but can be also formed using other lithography techniques, e.g., photolithography.

Aspects

The following Aspects are illustrative only and do not serve to limit the scope of the present disclosure or the appended claims.

Aspect 1. A microfluidic chip, comprising: a first supply channel configured to communicate a first fluid therein in a direction from upstream to downstream; a plurality of first delivery channels in fluid communication with the first supply channel; a second supply channel configured to communicate a second fluid therein in a direction from upstream to downstream; a plurality of second delivery channels in fluid communication with the second supply channel, a number of rows comprising a number of mixing device units, a mixing device unit comprising (i) a micromixer channel, (ii) a first flow resistor placing the micromixer channel of that mixing device unit into fluid communication with a first delivery channel associated with that mixing device unit, and (iii) a second flow resistor placing the micromixer channel of that mixing device unit into fluid communication with a second delivery channel associated with that mixing device unit; and an output channel, the output channel being in fluid communication with at least one of the mixing device units, and the output channel being configured to collect an output of at least one of the mixing device units.

In some embodiments, one or more of the first supply channel, one or more of the plurality of first delivery channels, the second supply channel, one or more of the second plurality of second delivery channels, and one or more of the rows of mixing device units lies in a plane. This is not a requirement, however, as one or more of the foregoing can lie in different planes or elevations relative to one another.

Aspect 2. The microfluidic chip of Aspect 1, wherein the plurality of first delivery channels is arranged in a laddered fashion. The first delivery channels can also be arranged in a herringbone or other fashion.

Aspect 3. The microfluidic chip of any one of Aspects 1 to 2, wherein the plurality of second delivery channels is arranged in a laddered fashion. The second delivery channels can also be arranged in a herringbone or other fashion.

Without being bound to any particular theory or embodiment, the first plurality of delivery channels can be arranged in a uniformly-spaced fashion, e.g., that each first delivery channel is disposed at an equal distance (along the first supply channel) from its neighboring first delivery channels. Likewise, each second delivery can be disposed at an equal distance (along the second supply channel) from its neighboring second delivery channels.

Aspect 4. The microfluidic chip of any one of Aspects 1 to 3, wherein the first supply channel and the second supply channel are oriented parallel or substantially parallel to one another.

Aspect 5. The microfluidic chip of any one of Aspects 1 to 4, wherein the plurality of first delivery channels and the plurality of second delivery channels are oriented parallel or substantially parallel to each another.

Aspect 6. The microfluidic chip of any one of Aspects 1 to 5, wherein a micromixer channel comprises a micromixer stage to effect mixing of first fluid and second fluid communicated to the micromixer channel.

Aspect 7. The microfluidic chip of Aspect 6, wherein the micromixer stage comprises one or more surface features configured to encourage mixing of first fluid and second fluid communicated to the micromixer channel. Suitable surface features include, e.g., herringbones, circles, curves, polygons, and the like.

Surface features can extend or protrude into a channel of a micromixer stage, e.g., herringbone protrusions that extend into the interior of a channel. Surface features can also be characterized as being etched into the surface of a flow channel, e.g., grooves, slots, or other features that are formed into the surface of a flow channel. Surface features can extend into a channel from any and all directions, e.g., from the ceiling of a channel, from the floor of a channel, and/or from an interior side surface of a channel.

A micromixer stage can include a periodic set of surface features, e.g., a first set of five herringbones that extend into a flow channel of the micromixer stage, with those five herringbones being adjacent to a second set of five herringbones, and so on. A set of surface features can include a plurality of individual features, e.g., a set of 2, 3, 4, 5, 6, 7, 8, 9, 10, 11, 12, 13, 14, 15, 16, 17, 18, 19, or even 20 features, such as an array of 10 ridges oriented at a first angle relative to a direction of flow within a channel followed by an array of 10 ridges oriented at a second angle relative to the direction of flow within the channel.

Measured relative to the surface from which it extends (or into which it extends), a surface features can be oriented at an angle of from 1 degree to 90 degrees, and all intermediate values. A micromixer channel can include surface features disposed on one or more surfaces of the channel, e.g., herringbone features disposed on the ceiling of the channel and wave features disposed on the floor of the channel. The channel can include opposed surfaces (e.g., ceiling and floor) that have mirrored surface features, e.g., herringbone features on the ceiling and floor of the channel.

Aspect 8. The microfluidic chip of Aspect 7, wherein the surface features comprise herringbone protrusions.

Aspect 9. The microfluidic chip of any one of Aspects 1 to 8, wherein the microfluidic chip defines a plane, and wherein (a) a via perpendicular to the plane or substantially perpendicular to the plane places the first supply channel into fluid communication with the plurality of first delivery channels, (b) a via perpendicular to the plane or substantially perpendicular to the plane places the second supply channel into fluid communication with the plurality of second delivery channels, or both (a) and (b).

Aspect 10. The microfluidic chip of any one of Aspects 1 to 9, wherein (a) the microfluidic chip defines a plane and wherein the microfluidic chip defines a plurality of flow layers parallel to the plane, a flow layer comprising a number of rows comprising a number of mixing device units therein, and (b) optionally wherein at least one of the first supply channel and the second supply channel is in fluid communication with two of the plurality of flow layers. Without being bound to any particular theory or embodiment, a microfluidic chip can thus include two or more stacked layers, with each layer including components (e.g., mixing device units) that form the desired product. In this way, a microfluidic chip can provide for parallel fabrication of product at multiple levels simultaneously, as each stacked layer can be fed by one or more supply channels that feed other of the stacked layers. With this approach, a single supply channel can provide input material to hundreds or even thousands (or tens of thousands) of mixing device units that are all located on the same microfluidic chip.

As explained elsewhere herein, the disclosed devices can give rise to uniform mixing of reagents in mixing device units on the chip. For example, when two different dyes are introduced to a disclosed chip in a 1:1 (equal) ratio, the ratio of those dyes in the mixing units on the chip can be uniform (i.e., 1:1) or nearly uniform. As an example, the ratio of the foregoing dyes (introduced in an equal ratio to the chip) in a given mixing unit on the chip can be within 5%, 4%, 3%, 2%, or even within 1% of the average dye ratio for all mixing units on the chip.

A chip can include a plurality of layers, e.g., 1, 2, 3, 4, 5, 6, 7, 8, 9, 10, or more layers. Adjacent layers can be of the same thickness or define features of the same or similar heights, but this is not a requirement. As shown in example FIG. 13, a channel (e.g., a first delivery channel, a second delivery channel, a supply channel, a micromixer channel) can have a cross-sectional dimension (e.g., a height, a width) of, e.g., about 200 μm (e.g., from about 10 to about 1000 μm, from about 20 to about 900 μm, from about 30 to about 800 μm, from about 40 to about 700 μm, from about 50 to about 600 μm, from about 60 to about 500 μm, from about 70 to about 400 μm, from about 80 to about 300 μm, from about 90 to about 200 μm, or even from about 100 to about 150 μm and all intermediate values and sub-ranges. A resistor can have a cross-sectional dimension of from about 10 μm to about 100 μm, from about 20 μm to about 90 μm, from about 30 μm to about 80 μm, from about 40 to about 70 μm, or even from about 50 to about 60 μm, including all intermediate values and subranges.

A via can have a cross-sectional dimension (e.g., a height, a width) of, e.g., about 200 μm (e.g., from about 10 to about 1000 μm, from about 20 to about 900 μm, from about 30 to about 800 μm, from about 40 to about 700 μm, from about 50 to about 600 μm, from about 60 to about 500 μm, from about 70 to about 400 μm, from about 80 to about 300 μm, from about 90 to about 200 μm, or even from about 100 to about 150 μm and all intermediate values and sub-ranges.

A mixing channel (also termed a micromixer channel, in some instances) can have a cross-sectional dimension (e.g., a height, a width) of, e.g., about 200 μm (e.g., from about 10 to about 1000 μm, from about 20 to about 900 μm, from about 30 to about 800 μm, from about 40 to about 700 μm, from about 50 to about 600 μm, from about 60 to about 500 μm, from about 70 to about 400 μm, from about 80 to about 300 μm, from about 90 to about 200 μm, or even from about 100 to about 150 μm and all intermediate values and sub-ranges.

A micromixer (e.g., a herringbone feature) can have a cross-sectional dimension of from about 5 μm (or about 10 μm) to about 100 μm, from about 20 μm to about 90 μm, from about 30 μm to about 80 μm, from about 40 to about 70 μm, or even from about 50 to about 60 μm, including all intermediate values and subranges. An output channel can have a cross-sectional dimension (e.g., a height, a width) of, e.g., about 200 μm (e.g., from about 10 to about 1000 from about 20 to about 900 from about 30 to about 800 from about 40 to about 700 from about 50 to about 600 from about 60 to about 500 from about 70 to about 400 from about 80 to about 300 from about 90 to about 200 or even from about 100 to about 150 μm and all intermediate values and sub-ranges. As shown, individual features (e.g., channels, micromixers) can be formed in the same device layer, but can also be formed in different device layers.

Aspect 11. The microfluidic chip of any one of Aspects 1 to 10, wherein the first supply channel, the plurality of first delivery channels, the number of rows, and the mixing device units are configured such that, during operation, each mixing device unit receives essentially the same flow rate of the first fluid delivered at essentially the same pressure.

Aspect 12. The microfluidic chip of any one of Aspects 1 to 11, wherein the second supply channel, the plurality of second delivery channels, the number of rows, and the mixing device units are configured such that, during operation, each mixing device unit receives essentially the same flow rate of the second fluid delivered at essentially the same pressure.

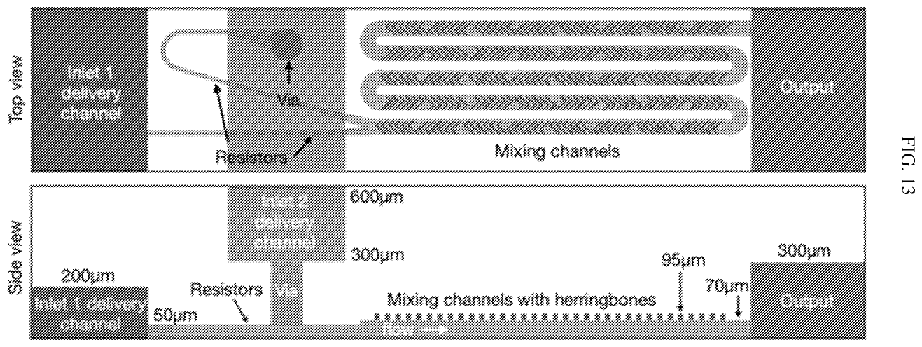

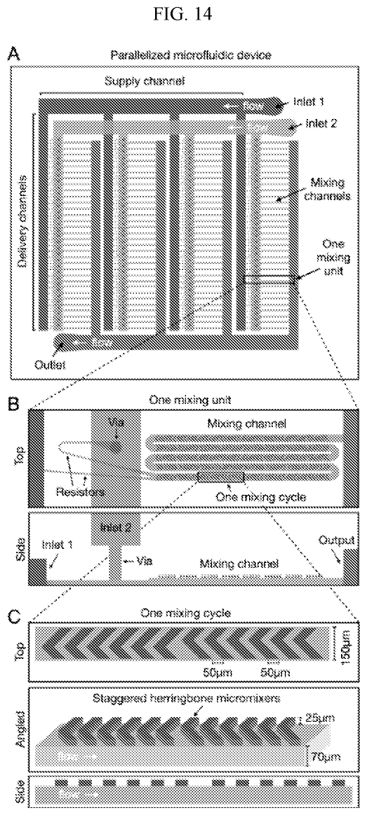

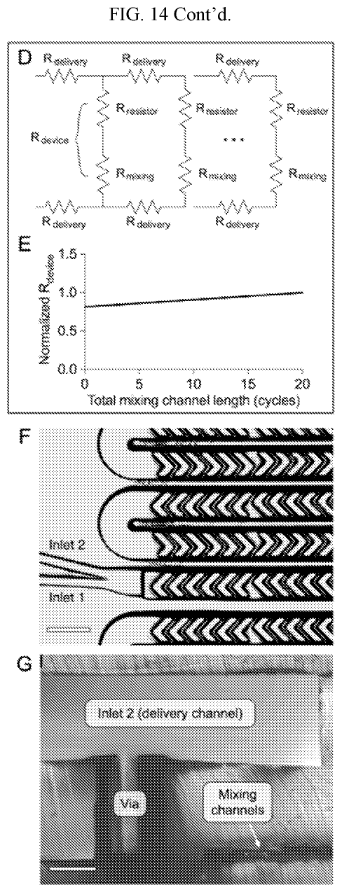

Aspect 13. The microfluidic chip of any one of Aspects 1 to 12, wherein
(a) a given mixing device unit defines a first fluidic resistance R_{device1} defined as the sum of (a) the fluidic resistance (which can be termed Rᵣₑₛᵢₛₜₒᵣ, by reference to FIG. 12) of the first flow resistor placing that mixing device unit into fluid communication with a first delivery channel associated with that mixing device unit and (b) the fluidic resistance (which can be termed R_{mixing}, by reference to FIG. 12) of the micromixer channel associated with that mixing device unit, wherein the first delivery channel defines a first fluidic resistance R_{delivery1} defined as the resistance of the portion of the first delivery channel that extends between the given mixing device unit and a mixing device unit located immediately upstream on the first delivery channel from the given mixing unit, and wherein a guideline number of mixing device units N_{device1} in a given row of mixing device units is defined by 2N_{device1}(R_{delivery}/R_{device1})<0.01,
(b) the given mixing device unit defines a second fluidic resistance R_{device2} defined as the sum of (a) the fluidic resistance of the second flow resistor placing that mixing device unit into fluid communication with the second delivery channel associated with that mixing device unit and (b) the fluidic resistance of the micromixer channel associated with that mixing device unit, wherein the second delivery channel defines a second fluidic resistance R_{delivery2} defined as the resistance of the portion of the second delivery channel that extends between the given mixing device unit and a mixing device unit located immediately upstream on the second delivery channel from the given mixing unit, and wherein a guideline number of mixing device units N_{device2} in the given row of mixing device units is defined by 2N_{device2}(R_{delivery2}/R_{device2})<0.01, and
(c) wherein the number of mixing devices in the given row is less than or equal to the lesser of N_{device1} and N_{device2}.

The foregoing is illustrated in FIG. 12, which provides a labeled version of FIG. 2A. As shown in FIG. 12, R_{delivery1} is the flow resistance (for the first fluid) of a delivery channel between a given mixing device unit and the mixing device unit that is immediately upstream (on the delivery channel) from the given mixing unit. R_{device1} is the flow resistance (for the first fluid) of a given mixing device unit, which flow resistance is comprised of the flow resistance Rᵣₑₛᵢₛₜₒᵣ of the first resistor (i.e., the resistor that receives the first fluid) and the flow resistance R_{mixing} of the micromixer channel of that mixing device unit.

Aspect 14. The microfluidic chip of Aspect 13, wherein
(a) a first fluidic resistance R_{row1} of a given row of mixing device units associated with a first fluid delivery channel DC₁ and a second fluid delivery channel DC₂ is defined by R_{row1}=R_{device1}/N_{device1}, a fluidic resistance R_{supply1} is defined by the fluidic resistance of the portion of the first supply channel (termed R_{supply} by reference to FIG. 12) that extends between (i) DC₁ and (ii) a first fluid delivery channel that is located immediately upstream on the first supply channel from DC₁, and a guideline number of rows N_{row1} associated with DC₁ is defined by 2N_{row1}(R_{supply1}/R_{row1})<0.01,
(b) a second fluidic resistance R_{row2} of the given row of mixing device units is defined by R_{row2}=R_{device2}/N_{device2}, a fluidic resistance R_{supply2} is defined by the fluidic resistance of the portion of the second supply channel that extends between (i) DC₂ and (ii) a second fluid delivery channel that is located immediately upstream on the second supply channel from DC₂, and a guideline number of rows N_{row2} associated with DC₂ is defined by 2N_{row2}(R_{supply2}/R_{row2})<0.01, and
(c) wherein the number of rows associated with the first fluid delivery channel and the second fluid delivery channel is less than or equal to the lesser of N_{row1} and N_{row2}.

The foregoing is illustrated by FIG. 12, which illustrates that R_{supply1} is the fluidic resistance of the portion of the first supply channel that extends between a given first delivery channel and a first fluid delivery channel that is located immediately upstream on the first supply channel from DC₁.

Aspect 15. The microfluidic chip of any one of Aspects 1 to 14, wherein the microfluidic chip comprises from 2 to 500,000 mixing device units. As an example, a microfluidic chip that is 3 inches by 3 inches in size can have 100 mixing device units. A microfluidic chip that is 4 inches by 4 inches can have more than 100 mixing device units. A microfluidic chip can have, e.g., from 2 to 100 mixing device units per square inch of the microfluidic chip.

Aspect 16. The microfluidic chip of any one of Aspects 1 to 15, wherein the microfluidic chip comprises from 2 to 100,000 rows of mixing device units.

Aspect 17. The microfluidic chip of any one of Aspects 1 to 16, wherein one or more of the first supply channel, the second supply channel, the first plurality of delivery channels, the second plurality of delivery channels, the output channel, or a mixing device unit is formed in silicon or glass.

It should be understood that a microfluidic chip according to the present disclosure can include more than two supply channels. As an example, a microfluidic chip according to the present disclosure can include, e.g., a third supply channel that carries a third fluid. Such a third supply channel can in turn be in fluid communication with a plurality of third fluid delivery channels, which third fluid delivery channels can in turn be in fluid communication (e.g., via third fluid resistors) with mixing unit devices associated with those third fluid delivery channels, the mixing unit devices also being in fluid communication with the output channel. The parallelized, modular architecture described herein allows for the use of one, two, three, four, or more supply channels, which supply channels can provide one, two, three, four, or more fluids to the mixing unit devices. In this way, a user can formulate products from one, two, three, four, or more fluids at high throughput. In some embodiments, the disclosed microfluidic chips can include 10s, 100s, or even 1000s of supply channels.

In some example embodiments, one can pre-mix a number of lipid excipients (e.g., 2-5) that are introduced to the device through a single input, with a medicament introduced through a second input. One can, however, also have an input for each of the lipid components if that is of use to a specific application. Similarly, if one wishes to incorporate multiple RNAs or DNAs into a single formulation, one can pre-mix those before they are introduced to the device, or one could have a separate input for each different type of nucleic acid/therapeutic. Thus, the disclosed architecture allows for pre-mixing of inputs before those pre-mixed inputs are themselves further mixed, as well as for introduction of separate inputs separately such that the separately-introduced inputs are mixed on the microfluidic chip.

Without being bound to any particular theory or embodiment, first and second delivery channels can be used to deliver fluids that are mixed to form LNPs, and the third delivery channel can be used to deliver a fluid that comprises one or more additional LNP components, e.g., a targeting ligand and/or a DNA/RNA barcode. A targeting ligand can be used to enable target specificity of the LNPs to different cells and tissues in the body, and DNA/RNA barcodes can be used to enable high-throughput screening of libraries of LNPs directly in the body. Thus, the present disclosure should be understood to include microfluidic chips that include more than two supply channels, as well as methods (described elsewhere herein) of operating such chips so as to give rise to products (e.g., LNPs) that comprise components provided by the specific fluids carried by each supply channel.

Aspect 18. The microfluidic chip of any one of claims 1 to 17, further comprising a third supply channel, the third supply channel being configured to communicate a third fluid in a direction from upstream to downstream, a plurality of third delivery channels in fluid communication with the third supply channel, a third delivery channel being in fluid communication with a mixing device unit that is associated with that third delivery channel and is in fluid communication with one or both of a first delivery channel and a second delivery channel.

Aspect 19. A method, comprising operating a microfluidic chip according to any one of Aspects 1 to 18 so as to give rise to an encapsulating product. The encapsulating product can be hollow, e.g., being capable of containing a species therein or thereon. One such example is a lipid nanoparticle having no other species disposed therein or thereon when the lipid nanoparticle is produced. Alternatively, the encapsulating product can include one or more species disposed therein, e.g., medicament, a label, a marker, and the like. Example medicaments include, e.g., vaccines, therapeutics, analgesics, biological molecules (such as nucleic acids and proteins) and the like. It should be understood that the present technology is not limited to the production of LNPs, and it should be understood that the present technology can be used to form a range of encapsulating products, including multilayered nanoparticles, such as core-shell structures.

Aspect 20. The method of Aspect 19, wherein the operating is performed such that the first supply channel, the plurality of first delivery channels, the number of rows, and the mixing device units are configured such that, during operation, each mixing device unit receives essentially the same flow rate of the first fluid delivered at essentially the same pressure.

Aspect 21. The method of any one of Aspects 19 to 20, wherein the operating is performed such that the second supply channel, the plurality of second delivery channels, the number of rows, and the mixing device units are configured such that, during operation, each mixing device unit receives essentially the same flow rate of the second fluid delivered at essentially the same pressure.

The disclosed technology can be used to form a variety of products, including (without limitation) lipid nanoparticles, lipid/polymer nanoparticles, liposomes, polymersomes, and the like, into any of which a therapeutic can be enclosed. As described elsewhere herein, the disclosed technology can also be used to encapsulate a therapeutic molecule or other contents, including (without limitation), nucleic acids, drugs (including proteins and biologics), genes/gene therapy molecules, and the like. Drugs can be small molecules (e.g., <900 Da), but can also be biologics like proteins and peptides (e.g., >900 Da to 100's of kDa).

Aspect 22. The method of any one of Aspects 19 to 21, wherein the encapsulating product defines a cross-sectional dimension in the range of 20 to about 1000 nm, including all values and subranges.

Aspect 23. The method of any one of Aspects 19 to 22, wherein the encapsulating product comprises a medicament. Example medicaments include, without limitation, a nucleic acid (including RNAs and DNAs), a drug (including small molecules, proteins, and biologics), a vaccine, genes/gene therapy molecules, and the like, as the disclosed methods are suitable to encapsulate essentially any species.

Aspect 24. The method of Aspect 23, wherein the medicament comprises a nucleic acid (including RNAs and DNAs), a drug, a vaccine, or any combination thereof. A drug can be, e.g., any small molecule, protein, biologic, and the like.

Aspect 25. The method of Aspect 23, wherein the encapsulating product comprises a medicament that is encapsulated within a lipid nanoparticle, within a polymer nanoparticle, within a protein-based nanoparticle, or within any combination thereof.

Aspect 26. The method of Aspect 25, wherein the medicament comprises messenger RNA (mRNA), small interfering RNA (siRNA), microRNA, antisense oligonucleotides, plasmid DNA, DNA oligonucleotides, one or more Cas9 proteins, circular RNAs, RNA replicons, self-amplifying RNAs, DNA barcodes, RNA barcodes, or any combination thereof. It should be understood that the disclosed technology can also encapsulate an additional species (e.g., small molecule, protein, or biologic) into an LNP or other encapsulating product along with an mRNA or any of the foregoing. Without being bound to any particular theory, the inclusion in the additional species can boost the therapy.

Aspect 27. The method of Aspect 26, wherein the mRNA is effective to vaccinate against an infectious disease. As but one example, the mRNA can be an mRNA effective to vaccinate against a coronavirus.

Aspect 28. The method of Aspect 27, wherein the mRNA is effective to vaccinate against a coronavirus. In addition to vaccines, mRNA can be effective for (1) protein replacement therapies for a range of diseases; and also (2) genome editing therapies, e.g., for monogenic disorders.

Aspect 29. The method of claim 26, wherein the mRNA is effective as a protein replacement therapy.

Aspect 30. The method of claim 26, wherein the mRNA is effective in genome editing.

### REFERENCES

The following references are provided for the reader's convenience; the listing of a reference here is not necessarily an indication that a reference is material to the patentability of the disclosed technology.
(1) Abrams, M. T.; Koser, M. L.; Seitzer, J.; Williams, S. C.; Dipietro, M. A.; Wang, W.; Shaw, A. W.; Mao, X.; Jadhav, V.; Davide, J. P.; Burke, P. A.; Sachs, A. B.; Stirdivant, S. M.; Sepp-Lorenzino, L. Evaluation of Efficacy, Biodistribution, and Inflammation for a Potent SiRNA Nanoparticle: Effect of Dexamethasone Co-Treatment. Mol. Ther. 2010, 18 (1), 171-180. https://doi.org/10.1038/mt.2009.208.
(2) Crawford, R.; Dogdas, B.; Keough, E.; Haas, R. M.; Wepukhulu, W.; Krotzer, S.; Burke, P. A.; Sepp-Lorenzino, L.; Bagchi, A.; Howell, B. J. Analysis of Lipid Nanoparticles by Cryo-EM for Characterizing SiRNA Delivery Vehicles. Int. J. Pharm. 2011, 403 (1-2), 237-244. https://doi.org/10.1016/j.ijpharm.2010.10.025.
(3) Jeffs, L. B.; Palmer, L. R.; Ambegia, E. G.; Giesbrecht, C.; Ewanick, S.; MacLachlan, I. A Scalable, Extrusion-Free Method for Efficient Liposomal Encapsulation of Plasmid DNA. Pharm. Res. 2005, 22 (3), 362-372. https://doi.org/10.1007/s11095-004-1873-z.
(4) Heyes, J.; Palmer, L.; Bremner, K.; MacLachlan, I. Cationic Lipid Saturation Influences Intracellular Delivery of Encapsulated Nucleic Acids. J. Control. Release 2005, 107 (2), 276-287. https://doi.org/10.1016/j.jconrel.2005.06.014.
(5) Zimmermann, T. S.; Lee, A. C. H.; Akinc, A.; Bramlage, B.; Bumcrot, D.; Fedoruk, M. N.; Harborth, J.; Heyes, J. A.; Jeffs, L. B.; John, M.; Judge, A. D.; Lam, K.; McClintock, K.; Nechev, L. V.; Palmer, L. R.; Racie, T.; Röhl, I.; Seiffert, S.; Shanmugam, S.; Sood, V.; Soutschek, J.; Toudjarska, I.; Wheat, A. J.; Yaworski, E.; Zedalis, W.; Koteliansky, V.; Manoharan, M.; Vornlocher, H. P.; MacLachlan, I. RNAi-Mediated Gene Silencing in Non-Human Primates. Nature 2006, 441 (1), 111-114. https://doi.org/10.1038/nature04688.
(6) Krzyszton, R.; Salem, B.; Lee, D. J.; Schwake, G.; Wagner, E.; Rädler, J. O. Microfluidic Self-Assembly of Folate-Targeted Monomolecular SiRNA-Lipid Nanoparticles. Nanoscale 2017, 9 (22), 7442-7453. https://doi.org/10.1039/c7nr01593c.
(7) Hood, R. R.; Devoe, D. L.; Atencia, J.; Vreeland, W. N.; Omiatek, D. M. A Facile Route to the Synthesis of Monodisperse Nanoscale Liposomes Using 3D Microfluidic Hydrodynamic Focusing in a Concentric Capillary Array. Lab Chip 2014, 14 (14), 2403-2409. https://doi.org/10.1039/c41c00334a.
(8) Belliveau, N. M.; Huft, J.; Lin, P. J.; Chen, S.; Leung, A. K.; Leaver, T. J.; Wild, A. W.; Lee, J. B.; Taylor, R. J.; Tam, Y. K.; Hansen, C. L.; Cullis, P. R. Microfluidic Synthesis of Highly Potent Limit-Size Lipid Nanoparticles for in Vivo Delivery of SiRNA. Mol. Ther.—Nucleic Acids 2012, 1 (8), e37. https://doi.org/10.1038/mtna.2012.28.
(9) Chen, D.; Love, K. T.; Chen, Y.; Eltoukhy, A. A.; Kastrup, C.; Sahay, G.; Jeon, A.; Dong, Y.; Whitehead, K. A.; Anderson, D. G. Rapid Discovery of Potent SiRNA-Containing Lipid Nanoparticles Enabled by Controlled Microfluidic Formulation. J. Am. Chem. Soc. 2012, 134 (16), 6948-6951. https://doi.org/10.1021/ja301621z.
(10) Webb, C.; Forbes, N.; Roces, C. B.; Anderluzzi, G.; Lou, G.; Abraham, S.; Ingalls, L.; Marshall, K.; Leaver, T. J.; Watts, J. A.; Aylott, J. W.; Perrie, Y. Using Microfluidics for Scalable Manufacturing of Nanomedicines from Bench to GMP: A Case Study Using Protein-Loaded Liposomes. Int. J. Pharm. 2020, 582 (April), 119266. https://doi.org/10.1016/j.ijpharm.2020.119266.
(11) Burnett, J. C.; Rossi, J. J. RNA-Based Therapeutics: Current Progress and Future Prospects. Chem. Biol. 2012, 19 (1), 60-71. https://doi.org/10.1016/j.chembiol.2011.12.008.
(12) Setten, R. L.; Rossi, J. J.; Han, S. ping. The Current State and Future Directions of RNAi-Based Therapeutics. Nat. Rev. Drug Discov. 2019, 18 (June), 421-446. https://doi.org/10.1038/s41573-019-0017-4.
(13) Patel, S.; Ryals, R. C.; Weller, K. K.; Pennesi, M. E.; Sahay, G. Lipid Nanoparticles for Delivery of Messenger RNA to the Back of the Eye. J. Control. Release 2019, 303 (March), 91-100. https://doi.org/10.1016/j.jconrel.2019.04.015.
(14) Yin, H.; Kanasty, R. L.; Eltoukhy, A. A.; Vegas, A. J.; Dorkin, J. R.; Anderson, D. G. Non-Viral Vectors for Gene-Based Therapy. Nat. Rev. Genet. 2014, 15, 541-555. https://doi.org/10.1038/nrg3763.
(15) Semple, S. C.; Akinc, A.; Chen, J.; Sandhu, A. P.; Mui, B. L.; Cho, C. K.; Sah, D. W. Y.; Stebbing, D.; Crosley, E. J.; Yaworski, E.; Hafez, I. M.; Dorkin, J. R.; Qin, J.; Lam, K.; Raj eev, K. G.; Wong, K. F.; Jeffs, L. B.; Nechev, L.; Eisenhardt, M. L.; Jayaraman, M.; Kazem, M.; Maier, M. A.; Srinivasulu, M.; Weinstein, M. J.; Chen, Q.; Alvarez, R.; Barros, S. A.; De, S.; Klimuk, S. K.; Borland, T.; Kosovrasti, V.; Cantley, W. L.; Tam, Y. K.; Manoharan, M.; Ciufolini, M. A.; Tracy, M. A.; De Fougerolles, A.; MacLachlan, I.; Cullis, P. R.; Madden, T. D.; Hope, M. J. Rational Design of Cationic Lipids for SiRNA Delivery. Nat. Biotechnol. 2010, 28 (2), 172-176. https://doi.org/10.1038/nbt.1602.
(16) Chen, G.; Abdeen, A. A.; Wang, Y.; Shahi, P. K.; Robertson, S.; Xie, R.; Suzuki, M.; Pattnaik, B. R.; Saha, K.; Gong, S. A Biodegradable Nanocapsule Delivers a Cas9 Ribonucleoprotein Complex for in Vivo Genome Editing. Nat. Nanotechnol. 2019, 14 (10), 974-980. https://doi.org/10.1038/s41565-019-0539-2.
(17) Debacker, A. J.; Voutila, J.; Catley, M.; Blakey, D.; Habib, N. Delivery of Oligonucleotides to the Liver with GalNAc: From Research to Registered Therapeutic Drug. Mol. Ther. 2020, 28 (8), 1759-1771. https://doi.org/10.1016/j.ymthe.2020.06.015.
(18) Barba, A. A.; Bochicchio, S.; Dalmoro, A.; Lamberti, G. Lipid Delivery Systems for Nucleic-Acid-Based-Drugs: From Production to Clinical Applications. Pharmaceutics 2019, 11 (8), 5-7. https://doi.org/10.3390/pharmaceutics11080360.
(19) Clinicaltrials.gov. A Study to Evaluate Efficacy, Safety, and Immunogenicity of mRNA-1273 Vaccine in Adults Aged 18 Years and Older to Prevent COVID-19 https://clinicaltrials.gov/ct2/show/study/NCT04470427.
(20) Clinicaltrials.gov. Safety and Immunogenicity of SARS-CoV-2 mRNA Vaccine (BNT162b1) in Chinese Healthy Subjects https://clinicaltrials.gov/ct2/show/NCT04523571?term=mrna&cond=Covid19&dra w=2&rank=4.
(21) Clinicaltrials.gov. A Dose-Confirmation Study to Evaluate the Safety, Reactogenicity and Immunogenicity of Vaccine CVnCoV in Healthy Adults https://clinicaltrials.gov/ct2/show/NCT04515147?term=mrna&cond=Covid19&dra w=2&rank=6.
(22) Clinicaltrials.gov. Ascending Dose Study of Investigational SARS-CoV-2 Vaccine ARCT-021 in Healthy Adult Subjects https://clinicaltrials.gov/ct2/show/NCT04480957?term=mrna&cond=Covid19&dra w=3&rank=13.
(23) Jackson, L. A.; Anderson, E. J.; Rouphael, N. G.; Roberts, P. C.; Makhene, M.; Coler, R. N.; McCullough, M. P.; Chappell, J. D.; Denison, M. R.; Stevens, L. J.; Pruijssers, A. J.; McDermott, A.; Flach, B.; Doria-Rose, N. A.; Corbett, K. S.; Morabito, K. M.; O'Dell, S.; Schmidt, S. D.; Swanson, P. A.; Padilla, M.; Mascola, J. R.; Neuzil, K. M.; Bennett, H.; Sun, W.; Peters, E.; Makowski, M.; Albert, J.; Cross, K.; Buchanan, W.; Pikaart-Tautges, R.; Ledgerwood, J. E.; Graham, B. S.; Beigel, J. H. An MRNA Vaccine against SARS-CoV-2—Preliminary Report. N. Engl. J. Med. 2020. https://doi.org/10.1056/nejmoa2022483.
(24) Mulligan, M. J.; Lyke, K. E.; Kitchin, N.; Absalon, J.; Gurtman, A.; Lockhart, S.; Neuzil, K.; Raabe, V.; Bailey, R.; Swanson, K. A.; Li, P.; Koury, K.; Kalina, W.; Cooper, D.; Fontes-Garfias, C.; Shi, P.-Y.; Tureci, O.; Tompkins, K. R.; Walsh, E. E.; Frenck, R.; Falsey, A. R.; Dormitzer, P. R.; Gruber, W. C.; Sahin, U.; Jansen, K. U. Phase 1/2 Study to Describe the Safety and Immunogenicity of a COVID-19 RNA Vaccine Candidate (BNT162b1) in Adults 18 to 55 Years of Age: Interim Report. medRxiv 2020, 1-16. https://doi.org/https://doi.org/10.1101/2020.06.30.20142570.
(25) Guan, S.; Rosenecker, J. Nanotechnologies in Delivery of MRNA Therapeutics Using Nonviral Vector-Based Delivery Systems. Gene Ther. 2017, 24 (3), 133-143. https://doi.org/10.1038/gt.2017.5.
(26) Davidson, B. L.; McCray, P. B. Current Prospects for RNA Interference-Based Therapies. Nat. Rev. Genet. 2011, 12 (5), 329-340. https://doi.org/10.1038/nrg2968.
(27) Puri, A.; Loomis, K.; Smith, B.; Lee, J.-H.; Yavlovich, A.; Heldman, E.; Blumenthal, R. Lipid-Based Nanoparticles as Pharmaceutical Drug Carriers: From Concepts to Clinic. Crit Rev Ther Drug Carr. Syst. 2009, 26 (6), 523-580.
(28) Kulkarni, J. A.; Cullis, P. R.; Van Der Meel, R. Lipid Nanoparticles Enabling Gene Therapies: From Concepts to Clinical Utility. Nucleic Acid Ther. 2018, 28 (3), 146-157. https://doi.org/10.1089/nat.2018.0721.
(29) Akinc, A.; Maier, M. A.; Manoharan, M.; Fitzgerald, K.; Jayaraman, M.; Barros, S.; Ansell, S.; Du, X.; Hope, M. J.; Madden, T. D.; Mui, B. L.; Semple, S. C.; Tam, Y. K.; Ciufolini, M.; Witzigmann, D.; Kulkarni, J. A.; van der Meel, R.; Cullis, P. R. The Onpattro Story and the Clinical Translation of Nanomedicines Containing Nucleic Acid-Based Drugs. Nat. Nanotechnol. 2019, 14 (12), 1084-1087. https://doi.org/10.1038/s41565-019-0591-y.
(30) Kauffman, K. J.; Dorkin, J. R.; Yang, J. H.; Heartlein, M. W.; Derosa, F.; Mir, F. F.; Fenton, O. S.; Anderson, D. G. Optimization of Lipid Nanoparticle Formulations for MRNA Delivery in Vivo with Fractional Factorial and Definitive Screening Designs. Nano Lett. 2015, 15 (11), 7300-7306. https://doi.org/10.1021/acs.nanolett.5b02497.
(31) Ball, R. L.; Hajj, K. A.; Vizelman, J.; Bajaj, P.; Whitehead, K. A. Lipid Nanoparticle Formulations for Enhanced Co-Delivery of SiRNA and MRNA. Nano Lett. 2018, 18 (6), 3814-3822. https://doi.org/10.1021/acs.nanolett.8b01101.
(32) Love, K. T.; Mahon, K. P.; Levins, C. G.; Whitehead, K. A.; Querbes, W.; Dorkin, J. R.; Qin, J.; Cantley, W.; Qin, L. L.; Racie, T.; Frank-Kamenetsky, M.; Yip, K. N.; Alvarez, R.; Sah, D. W. Y.; de Fougerolles, A.; Fitzgerald, K.; Koteliansky, V.; Akinc, A.; Langer, R.; Anderson, D. G. Lipid-like Materials for Low-Dose, in Vivo Gene Silencing. Proc. Natl. Acad. Sci. 2010, 107 (5), 1864-1869. https://doi.org/10.1073/pnas.0910603106.
(33) Evers, M. J. W.; Kulkarni, J. A.; van der Meel, R.; Cullis, P. R.; Vader, P.; Schiffelers, R. M. State-of-the-Art Design and Rapid-Mixing Production Techniques of Lipid Nanoparticles for Nucleic Acid Delivery. Small Methods 2018, 2 (9), 1700375. https://doi.org/10.1002/smtd.201700375.
(34) Garg, S.; Heuck, G.; Ip, S.; Ramsay, E. Microfluidics: A Transformational Tool for Nanomedicine Development and Production. J. Drug Target. 2016, 24 (9), 821-835. https://doi.org/10.1080/1061186X.2016.1198354.
(35) Maurer, N.; Wong, K. F.; Stark, H.; Louie, L.; McIntosh, D.; Wong, T.; Scherrer, P.; Semple, S. C.; Cullis, P. R. Spontaneous Entrapment of Polynucleotides upon Electrostatic Interaction with Ethanol-Destabilized Cationic Liposomes. Biophys. J. 2001, 80 (5), 2310-2326. https://doi.org/10.1016/S0006-3495(01)76202-9.
(36) Valencia, P.; Farokhzad, O.; Karnik, R.; Langer, R. Microfluidic Technologies for Accelerating the Clinical Translation of Nanoparticles. Nat. Nanotechnol. 2012, 7, 623-629. https://doi.org/10.1038/NNANO.2012.168.
(37) Jahn, A.; Vreeland, W. N.; Gaitan, M.; Locascio, L. E. Controlled Vesicle Self-Assembly in Microfluidic Channels with Hydrodynamic Focusing. J. Am. Chem. Soc. 2004, 126 (9), 2674-2675. https://doi.org/10.1021/ja0318030.
(38) Jahn, A.; Stavis, S. M.; Hong, J. S.; Vreeland, W. N.; Devoe, D. L.; Gaitan, M. Microfluidic Mixing and the Formation of Nanoscale Lipid Vesicles. ACS Nano 2010, 4 (4), 2077-2087. https://doi.org/10.1021/nn901676x.
(39) Stroock, A. D.; Dertinger, S. K. W.; Ajdari, A.; Mezic, I.; Stone, H. A.; Whitesides, G. M. Chaotic Mixer for Microchannels. Science (80-.). 2002, 295, 647-651.
(40) Kis, Z.; Kontoravdi, C.; Shattock, R.; Shah, N. Resources, Production Scales and Time Required for Producing RNA Vaccines for the Global Pandemic Demand. Vaccines 2021, 9 (1), 1-14. https://doi.org/10.3390/vaccines9010003.
(41) Yadavali, S.; Jeong, H. H.; Lee, D.; Issadore, D. Silicon and Glass Very Large Scale Microfluidic Droplet Integration for Terascale Generation of Polymer Microparticles. Nat. Commun. 2018, 9 (1). https://doi.org/10.1038/s41467-018-03515-2.
(42) Yadavali, S.; Lee, D.; Issadore, D. Robust Microfabrication of Highly Parallelized Three-Dimensional Microfluidics on Silicon. Sci. Rep. 2019, 9 (1), 1-10. https://doi.org/10.1038/s41598-019-48515-4.
(43) Romanowsky, M. B.; Abate, A. R.; Rotem, A.; Holtze, C.; Weitz, D. A. High Throughput Production of Single Core Double Emulsions in a Parallelized Microfluidic Device. Lab Chip 2012, 12 (4), 802-807. https://doi.org/10.1039/c21c21033a.
(44) Muluneh, M.; Issadore, D. Hybrid Soft-Lithography/Laser Machined Microchips for the Parallel Generation of Droplets. Lab Chip 2013, 13 (24), 4750-4754. https://doi.org/10.1039/c31c50979f.
(45) Jeong, H. H.; Yelleswarapu, V. R.; Yadavali, S.; Issadore, D.; Lee, D. Kilo-Scale Droplet Generation in Three-Dimensional Monolithic Elastomer Device (3D MED). Lab Chip 2015, 15 (23), 4387-4392. https://doi.org/10.1039/c51c01025j.
(46) Tsao, C. W. Polymer Microfluidics: Simple, Low-Cost Fabrication Process Bridging Academic Lab Research to Commercialized Production. Micromachines 2016, 7 (12). https://doi.org/10.3390/mi7120225.
(47) Ng Lee, J.; Park, C.; Whitesides, G. M. Solvent Compatibility of Poly(Dimethylsiloxane)-Based Microfluidic Devices. Anal. Chem. 2003, 75 (23), 6544-6554. https://doi.org/10.1021/ac0346712.
(48) Leung, A. K. K.; Hafez, I. M.; Baoukina, S.; Belliveau, N. M.; Zhigaltsev, I. V.; Afshinmanesh, E.; Tieleman, D. P.; Hansen, C. L.; Hope, M. J.; Cullis, P. R. Lipid Nanoparticles Containing SiRNA Synthesized by Microfluidic Mixing Exhibit an Electron-Dense Nanostructured Core. J. Phys. Chem. C 2012, 116 (34), 18440-18450. https://doi.org/10.1021/jp303267y.
(49) Akinc, A.; Zumbuehl, A.; Goldberg, M.; Leshchiner, E. S.; Busini, V.; Hossain, N.; Bacallado, S. A.; Nguyen, D. N.; Fuller, J.; Alvarez, R.; Borodovsky, A.; Borland, T.; Constien, R.; De Fougerolles, A.; Dorkin, J. R.; Narayanannair Jayaprakash, K.; Jayaraman, M.; John, M.; Koteliansky, V.; Manoharan, M.; Nechev, L.; Qin, J.; Racie, T.; Raitcheva, D.; Rajeev, K. G.; Sah, D. W. Y.; Soutschek, J.; Toudjarska, I.; Vornlocher, H. P.; Zimmermann, T. S.; Langer, R.; Anderson, D. G. A Combinatorial Library of Lipid-like Materials for Delivery of RNAi Therapeutics. Nat. Biotechnol. 2008, 26 (5), 561-569. https://doi.org/10.1038/nbt1402.
(50) Ball, R.; Bajaj, P.; Whitehead, K. A. Achieving Long-Term Stability of Lipid Nanoparticles: Examining the Effect of PH, Temperature, and Lyophilization. Int. J. Nanomedicine 2017, 12, 305-315. https://doi.org/10.2147/IJN.S123062.
(51) Yin, H.; Song, C. Q.; Dorkin, J. R.; Zhu, L. J.; Li, Y.; Wu, Q.; Park, A.; Yang, J.; Suresh, S.; Bizhanova, A.; Gupta, A.; Bolukbasi, M. F.; Walsh, S.; Bogorad, R. L.; Gao, G.; Weng, Z.; Dong, Y.; Koteliansky, V.; Wolfe, S. A.; Langer, R.; Xue, W.; Anderson, D. G. Therapeutic Genome Editing by Combined Viral and Non-Viral Delivery of CRISPR System Components in Vivo. Nat. Biotechnol. 2016, 34 (3), 328-333. https://doi.org/10.1038/nbt.3471.
(52) Whitehead, K. A.; Dorkin, J. R.; Vegas, A. J.; Chang, P. H.; Veiseh, O.; Matthews, J.; Fenton, O. S.; Zhang, Y.; Olejnik, K. T.; Yesilyurt, V.; Chen, D.; Barros, S.; Klebanov, B.; Novobrantseva, T.; Langer, R.; Anderson, D. G. Degradable Lipid Nanoparticles with Predictable in Vivo SiRNA Delivery Activity. Nat. Commun. 2014, 5. https://doi.org/10.1038/ncomms5277.
(53) Oberli, M. A.; Reichmuth, A. M.; Dorkin, J. R.; Mitchell, M. J.; Fenton, O. S.; Jaklenec, A.; Anderson, D. G.; Langer, R.; Blankschtein, D. Lipid Nanoparticle Assisted MRNA Delivery for Potent Cancer Immunotherapy. Nano Lett. 2017, 17 (3), 1326-1335. https://doi.org/10.1021/acs.nanolett.6b03329.
(54) Billingsley, M. M.; Singh, N.; Ravikumar, P.; Zhang, R.; June, C. H.; Mitchell, M. J. Ionizable Lipid Nanoparticle-Mediated MRNA Delivery for Human CAR T Cell Engineering. Nano Lett. 2020, 20 (3), 1578-1589. https://doi.org/10.1021/acs.nanolett.9b04246.
(55) Petros, R. A.; Desimone, J. M. Strategies in the Design of Nanoparticles for Therapeutic Applications. Nat. Rev. Drug Discov. 2010, 9 (8), 615-627. https://doi.org/10.1038/nrd2591.
(56) Morton, S. W.; Herlihy, K. P.; Shopsowitz, K. E.; Deng, Z. J.; Chu, K. S.; Bowerman, C. J.; Desimone, J. M.; Hammond, P. T. Scalable Manufacture of Built-to-Order Nanomedicine: Spray-Assisted Layer-by-Layer Functionalization of PRINT Nanoparticles. Adv. Mater. 2013, 25 (34), 4707-4713. https://doi.org/10.1002/adma.201302025.
(57) Frenz, L.; El Harrak, A.; Pauly, M.; Bégin-Colin, S.; Griffiths, A. D.; Baret, J. C. Droplet-Based Microreactors for the Synthesis of Magnetic Iron Oxide Nanoparticles. Angew. Chemie—Int. Ed. 2008, 47 (36), 6817-6820. https://doi.org/10.1002/a nie.200801360.
(58) Abalde-Cela, S.; Taladriz-Blanco, P.; De Oliveira, M. G.; Abell, C. Droplet Microfluidics for the Highly Controlled Synthesis of Branched Gold Nanoparticles. Sci. Rep. 2018, 8 (1), 1-6. https://doi.org/10.1038/s41598-018-20754-x.
(59) Witzigmann, D.; Uhl, P.; Sieber, S.; Kaufman, C.; Einfalt, T.; Schoneweis, K.; Grossen, P.; Buck, J.; Ni, Y.; Schenk, S. H.; Hussner, J.; Meyer zu Schwabedissen, H. E.; Quebatte, G.; Mier, W.; Urban, S.; Huwyler, J. Optimization-by-Design of Hepatotropic Lipid Nanoparticles Targeting the Sodium-Taurocholate Cotransporting Polypeptide. Elife 2019, 8, 1-28. https://doi.org/10.7554/eLife.42276.
(60) Witzigmann, D.; Kulkarni, J. A.; Leung, J.; Chen, S.; Cullis, P. R.; van der Meel, R. Lipid Nanoparticle Technology for Therapeutic Gene Regulation in the Liver. Adv. Drug Deliv. Rev. 2020, 159, 344-363. https://doi.org/10.1016/j.addr.2020.06.026.
(61) Pardi, N.; Tuyishime, S.; Muramatsu, H.; Kariko, K.; Mui, B. L.; Tam, Y. K.; Madden, T. D.; Hope, M. J.; Weissman, D. Expression Kinetics of Nucleoside-Modified MRNA Delivered in Lipid Nanoparticles to Mice by Various Routes. J. Control. Release 2015, 217, 345-351. https://doi.org/10.1016/j.jconrel.2015.08.007.
(62) Otto, G. P.; Rathkolb, B.; Oestereicher, M. A.; Lengger, C. J.; Moerth, C.; Micklich, K.; Fuchs, H.; Gailus-durner, V.; Wolf, E.; de Angelis, M. H. Clinical Chemistry Reference Intervals for C57BL/6J, C57BL/6N, and C3HeB/FeJ Mice (Mus Musculus). J. Am. Assoc. Lab. Anim. Sci. 2016, 55 (4), 375-386.

1. A microfluidic chip, comprising:
a first supply channel configured to communicate a first fluid therein in a direction from upstream to downstream;
a plurality of first delivery channels in fluid communication with the first supply channel;
a second supply channel configured to communicate a second fluid therein in a direction from upstream to downstream;
a plurality of second delivery channels in fluid communication with the second supply channel,
a number of rows comprising a number of mixing device units,
a mixing device unit comprising (i) a micromixer channel, (ii) a first flow resistor placing the micromixer channel of that mixing device unit into fluid communication with a first delivery channel associated with that mixing device unit, and (iii) a second flow resistor placing the micromixer channel of that mixing device unit into fluid communication with a second delivery channel associated with that mixing device unit; and
an output channel,
the output channel being in fluid communication with at least one of the mixing device units, and
the output channel being configured to collect an output of at least one of the mixing device units.

2. The microfluidic chip of claim 1, wherein the plurality of first delivery channels is arranged in a laddered fashion.

3. The microfluidic chip of claim 1, wherein the plurality of second delivery channels is arranged in a laddered fashion.

4. The microfluidic chip of claim 1, wherein the first supply channel and the second supply channel are oriented parallel or substantially parallel to one another.

5. The microfluidic chip of claim 1, wherein the plurality of first delivery channels and the plurality of second delivery channels are oriented parallel or substantially parallel to each another.

6. The microfluidic chip of claim 1, wherein a micromixer channel comprises a micromixer stage to effect mixing of first fluid and second fluid communicated to the micromixer channel.

7. The microfluidic chip of claim 6, wherein the micromixer stage comprises one or more surface features configured to encourage mixing of first fluid and second fluid communicated to the micromixer channel.

8. The microfluidic chip of claim 7, wherein the surface features comprise herringbone protrusions.

9. The microfluidic chip of claim 1, wherein the microfluidic chip defines a plane, and wherein
(a) a via perpendicular to the plane or substantially perpendicular to the plane places the first supply channel into fluid communication with the plurality of first delivery channels,
(b) a via perpendicular to the plane or substantially perpendicular to the plane places the second supply channel into fluid communication with the plurality of second delivery channels, or
both (a) and (b).

10. The microfluidic chip of claim 1, wherein
(a) the microfluidic chip defines a plane and wherein the microfluidic chip defines a plurality of flow layers parallel to the plane, a flow layer comprising a number of rows comprising a number of mixing device units therein, and
(b) optionally wherein at least one of the first supply channel and the second supply channel is in fluid communication with two of the plurality of flow layers.

11. The microfluidic chip of claim 1, wherein the first supply channel, the plurality of first delivery channels, the number of rows, and the mixing device units are configured such that, during operation, each mixing device unit receives essentially the same flow rate of the first fluid delivered at essentially the same pressure.

12. The microfluidic chip of claim 1, wherein the second supply channel, the plurality of second delivery channels, the number of rows, and the mixing device units are configured such that, during operation, each mixing device unit receives essentially the same flow rate of the second fluid delivered at essentially the same pressure.

13. The microfluidic chip of claim 1, wherein
(a) a given mixing device unit defines a first fluidic resistance R_{device1} defined as the sum of (a) the fluidic resistance of the first flow resistor placing that mixing device unit into fluid communication with a first delivery channel associated with that mixing device unit and (b) the fluidic resistance of the micromixer channel associated with that mixing device unit,
wherein the first delivery channel defines a first fluidic resistance R_{delivery1} defined as the resistance of the portion of the first delivery channel that extends between the given mixing device unit and a mixing device unit located immediately upstream on the first delivery channel from the given mixing unit, and
wherein a guideline number of mixing device units N_{device1} in a given row of mixing device units is defined by 2N_{device1}(R_{delivery1}/R_{device1})<0.01,
(b) a mixing device unit defines a second fluidic resistance R_{device2} defined as the sum of (a) the fluidic resistance of the second flow resistor placing that mixing device unit into fluid communication with the second delivery channel associated with that mixing device unit and (b) the fluidic resistance of the micromixer channel associated with that mixing device unit,
wherein the second delivery channel defines a second fluidic resistance R_{delivery2} defined as the resistance of the portion of the second delivery channel that extends between the given mixing device unit and a mixing device unit located immediately upstream on the second delivery channel from the given mixing unit, and
wherein a guideline number of mixing device units N_{device2} in the given row of mixing device units is defined by 2N_{device2}(R_{delivery2}/R_{device2})<0.01, and
(c) wherein the number of mixing devices in the given row is less than or equal to the lesser of N_{device1} and N_{device2}.

14. The microfluidic chip of claim 13, wherein
(a) a first fluidic resistance R_{row1} of a given row of mixing device units associated with a first fluid delivery channel DC₁ and a second fluid delivery channel DC₂ is defined by R_{row1}=R_{device1}/N_{device1},
a fluidic resistance R_{supply1} is defined by the fluidic resistance of the portion of the first supply channel that extends between (i) DC₁ and (ii) a first fluid delivery channel that is located immediately upstream on the first supply channel from DC₁, and
a guideline number of rows N_{row1} associated with DC₁ is defined by 2N_{row1}(R_{supply1}/R_{row1})
<0.01,
(b) a second fluidic resistance R_{row2} of the given row of mixing device units is defined by R_{row2}=R_{device2}/N_{device2},
a fluidic resistance R_{supply2} is defined by the fluidic resistance of the portion of the second supply channel that extends between (i) DC₂ and (ii) a second fluid delivery channel that is located immediately upstream on the second supply channel from DC₂, and
a guideline number of rows N_{row2} associated with DC₂ is defined by 2N_{row2}(R_{supply2}/R_{row2})
<0.01, and
(c) wherein the number of rows associated with the first fluid delivery channel and the second fluid delivery channel is less than or equal to the lesser of N_{row1} and N_{row2}.

15. The microfluidic chip of claim 1, wherein the microfluidic chip comprises from 2 to 20,000 mixing device units.

16. The microfluidic chip of claim 1, wherein the microfluidic chip comprises from 2 to 1,000 rows of mixing device units.

17. The microfluidic chip of claim 1, wherein one or more of the first supply channel, the second supply channel, the first plurality of delivery channels, the second plurality of delivery channels, the output channel, or a mixing device unit is formed in silicon or glass.

18. The microfluidic chip of claim 1, further comprising a third supply channel, the third supply channel being configured to communicate a third fluid in a direction from upstream to downstream, a plurality of third delivery channels in fluid communication with the third supply channel, a third delivery channel being in fluid communication with a mixing device unit that is associated with that third delivery channel and is in fluid communication with one or both of a first delivery channel and a second delivery channel.

19. A method, comprising operating a microfluidic chip according to claim 1 so as to give rise to an encapsulating product.

20. The method of claim 19, wherein the operating is performed such that the first supply channel, the plurality of first delivery channels, the number of rows, and the mixing device units are configured such that, during operation, each mixing device unit receives essentially the same flow rate of the first fluid delivered at essentially the same pressure.

21. The method of claim 19, wherein the operating is performed such that the second supply channel, the plurality of second delivery channels, the number of rows, and the mixing device units are configured such that, during operation, each mixing device unit receives essentially the same flow rate of the second fluid delivered at essentially the same pressure.

22. The method of claim 19, wherein the encapsulating product defines a cross-sectional dimension in the range of 20 to about 1000 nm.

23. The method of claim 19, wherein the encapsulating product comprises a medicament.

24. The method of claim 23, wherein, wherein the medicament comprises a nucleic acid, a drug, a vaccine, or any combination thereof

25. The method of claim 24, wherein the encapsulating product comprises a medicament that is encapsulated within a lipid nanoparticle, within a polymer nanoparticle, within a protein-based nanoparticle, or within any combination thereof.

26. The method of claim 25, wherein the medicament comprises mRNA.

27. The method of claim 26, wherein the mRNA is effective to vaccinate against an infectious disease.

28. The method of claim 27, wherein the mRNA is effective to vaccinate against a coronavirus.

29. The method of claim 26, wherein the mRNA is effective as a protein replacement therapy.

30. The method of claim 26, wherein the mRNA is effective in genome editing.
